SEQUENCE LISTING (14 sequences)

SEQ ID NO: 1 - DNA, 21 bp, Artificial Sequence
  Synthetic oligonucleotide
cttcctcgatgcttctcttcc

SEQ ID NO: 2 - DNA, 17 bp, Artificial Sequence
  Synthetic oligonucleotide
gacctccagcgactcct

SEQ ID NO: 3 - DNA, 22 bp, Artificial Sequence
  Synthetic oligonucleotide
tgtaccgctatggttacactcg

SEQ ID NO: 4 - DNA, 19 bp, Artificial Sequence
  Synthetic oligonucleotide
ggcagggacagttgcttct

SEQ ID NO: 5 - DNA, 20 bp, Artificial Sequence
  Synthetic oligonucleotide
gcctccttagatcacagctc

SEQ ID NO: 6 - DNA, 21 bp, Artificial Sequence
  Synthetic oligonucleotide
gatgctcttcagttcgtgtgt

SEQ ID NO: 7 - DNA, 20 bp, Artificial Sequence
  Synthetic oligonucleotide
gcgctgtcatcgatttcttc

SEQ ID NO: 8 - DNA, 22 bp, Artificial Sequence
  Synthetic oligonucleotide
tcactcatggctttgtagatgc

SEQ ID NO: 9 - DNA, 23 bp, Artificial Sequence
  Synthetic oligonucleotide
caaaactcaaattggggtcacag

SEQ ID NO: 10 - DNA, 22 bp, Artificial Sequence
  Synthetic oligonucleotide
ctctctgctgatgacatacgtg

SEQ ID NO: 11 - DNA, 23 bp, Artificial Sequence
  Synthetic oligonucleotide
agctgaagataaatgcaagtgag

SEQ ID NO: 12 - DNA, 21 bp, Artificial Sequence
  Synthetic oligonucleotide
cagaacagctaaacggagtcg

SEQ ID NO: 13 - DNA, 19 bp, Artificial Sequence
  Synthetic oligonucleotide
tcataggggcaagacacct

SEQ ID NO: 14 - DNA, 22 bp, Artificial Sequence
  Synthetic oligonucleotide
gctgctcatcttactctttgtc

(19) United States
(12) Patent Application Publication
(10) Pub. No.: US 2021/0275583 A1
(43) Pub. Date: Sep. 9, 2021

(54) COMPOSITIONS AND METHODS FOR THE TREATMENT OF DEMYELINATING CONDITIONS

(71) Applicant: Duke University, Durham, NC (US)

(72) Inventors: Joanne Kurtzberg, Durham, NC (US); Anthony Fillano, Durham, NC (US); Arjun Saha, Durham, NC (US); Andrew E. Balber, Durham, NC (US); Ana Valverde, Durham, NC (US)

(73) Assignee: Duke University, Durham, NC (US)

(21) Appl. No.: 17/328,749

(22) Filed: May 24, 2021

Related U.S. Application Data
(63) Continuation-in-part of application No. 16/477,167, filed on Jul. 10, 2019.
(60) Provisional application No. 62/445,400, filed on Jan. 12, 2017.
(60) Provisional application No. 62/466,438, filed on Mar. 3, 2017.
(60) Provisional application No. 62/482,254, filed on Apr. 6, 2017.
(60) Provisional application No. 62/505,284, filed on May 12, 2017.

(51) Int. Cl. A61K 35/15; C12N 5/0786; C12N 5/079; A61K 35/30

(52) U.S. Cl. CPC A61K 35/15; C12N 5/0645; C12N 5/0622; C12N 2506/1369; C12N 2501/165; C12N 2501/135; A61K 35/30

(57) ABSTRACT

The present disclosure provides compositions and methods for treating demyelinating conditions. More particularly, the present disclosure relates to compositions comprising a DUOC-01 cell product; methods for preparing such compositions; and methods of using such compositions for treating demyelinating conditions.

### CROSS-REFERENCE TO RELATED APPLICATIONS

This application is a CIP of U.S. patent application Ser. No. 16/477,167, filed Jul. 10, 2019, which is the US national phase under 35 U.S.C. § 371 of International Application No. PCT/US2018/013606, filed Jan. 12, 2018, which claims the benefit of priority of U.S. Provisional Patent Application No. 62/445,400, filed Jan. 12, 2017, U.S. Provisional Patent Application No. 62/466,438, filed Mar. 3, 2017, U.S. Provisional Patent Application No. 62/482,254, Apr. 6, 2017, and U.S. Provisional Patent Application No. 62/505,284, May 12, 2017, all of which are incorporated herein by reference in their entireties.

### BACKGROUND OF THE INVENTION

### Field of the Invention

The present disclosure provides compositions and methods for the treatment of treating demyelinating conditions. More particularly, the present disclosure relates to compositions including DUOC-01 cell product; methods for preparing such compositions; and methods of using such compositions for treatment of treating demyelinating conditions.

### Description of the Related Art

Microglia play critical but incompletely understood roles in propagation and resolution of central nervous system (CNS) injuries. These cells modulate neuroinflammation, produce factors that regulate activities of astrocytes, oligodendrocytes, and neurons, and clear debris to provide an environment for oligodendrocytes to begin to remyelinate neurons. In mice, microglia arise from a unique pool of replicating precursors in the brain that is originally derived from the extraembryonic yolk sac early in fetal development. Bone marrow-derived, circulating blood monocytes constitute another potential source of infiltrating phagocytic cells that can exacerbate or ameliorate CNS damage. Although a pathway for circulation of monocytes between lymph and brain parenchyma has recently been described, large numbers of circulating monocytes do not enter the uninjured, adult mouse brain but may infiltrate the CNS following insult such as brain irradiation, chemotherapy or injury, demyelinating conditions, or chronic stress. In some models, these infiltrating blood monocytes may activate inflammation and participate in demyelinating events. In others, blood monocytes may facilitate remyelination.

Limited information is available concerning the role of human blood monocytes in the dynamics of repair of brain injury. Circulating human monocytes include subpopulations that differ in their ability to migrate to tissues, proliferate, and form inflammatory or reparative macrophages at sites of injury. Based on experiments in rodents, several groups have proposed that cell products composed of human monocytes could be considered as candidates for the treatment of injury-induced CNS demyelination (Shechter R, Schwartz M. J Pathol. 2013; 229(2):332-346; Sanberg P R, et al. J Cell Mol Med. 2010; 14(3):553-563). CD14+ monocytes present in human umbilical cord blood (CB) are among these candidates. CB mononuclear cells are protective in several in vitro culture and animal models of CNS injury (Sun J M, Kurtzberg J. Cytotherapy. 2015; 17(6):775-785), and CB CD14+ cells are essential for the protective ability of intravenously injected CB mononuclear cells in the rat middle cerebral artery occlusion model of stroke (Womble T A, et al. Mol Cell Neurosci. 2014; 59:76-84).

The inventors have developed DUOC-01, a cell therapy product composed of cells with characteristics of macrophages and microglia that is intended for use in the treatment of demyelinating CNS diseases. DUOC-01 is manufactured by culturing banked cryopreserved and thawed CB-derived mononuclear cells (MNCs) in adherent cell culture over 21 days. The motile, phagocytic cells in DUOC-01 express CD45, CD11b, CD14, CD16, CD206, ionized calcium binding adaptor molecule 1 (Iba1), HLA-DR, and iNOS, secrete IL-10 and IL-6, and upregulate secretion of anti-inflammatory cytokines both constitutively and in response to TNF-α and IFN-γ (Kurtzberg J, et al. Cytotherapy. 2015; 17(6):803-815). DUOC-01 cells derived from genetically normal umbilical cord blood donors also secrete a battery of lysosomal hydrolases that are missing in children with leukodystrophies. DUOC-01, administered intrathecally 1-2 months after an unrelated donor umbilical cord blood transplant, provides cross-correcting normal enzyme to slow neurodegeneration before definitive engraftment by wild-type enzyme-producing cells from the systemic CB transplant.

It has been currently determined that DUOC-01 cells have the potential for therapeutic use in demyelinating conditions as a stand-alone cellular product.

### SUMMARY OF THE INVENTION

One aspect of the present disclosure includes methods for treating demyelinating conditions. Such methods include administering to the subject in need thereof a therapeutically effective amount of a composition comprising a DUOC-01 cell product in a pharmaceutically acceptable carrier,
wherein the DUOC-01 cell product comprises cells derived from cord blood mononuclear cells, wherein such cells express one or more of CD45, CD11b, CD14, CD16, CD206, CD163, Iba1, HLA-DR, TREM 2, and iNOS macrophage or microglia markers; and wherein such cells secrete IL-6, IL-10, or both.

Demyelinating conditions include, but are not limited to, leukodystrophies, multiple sclerosis, spinal cord injury, peripheral nerve damage, Parkinson's disease, amyotrophic lateral sclerosis (ALS), and Alzheimer's disease.

In certain embodiments of the disclosure, the methods are for treating multiple sclerosis in a subject. In certain embodiments of the disclosure, the methods are for treating leukodystrophies in a subject. In certain embodiments of the disclosure, the methods are for treating spinal cord injury in a subject.

Another aspect of the disclosure provides methods for promoting local nerve regeneration. Such methods include administering to the subject in need thereof a therapeutically effective amount of a composition comprising a DUOC-01 cell product as described herein in a pharmaceutically acceptable carrier. For example, in certain embodiments, the disclosure provides methods for promoting local nerve regeneration after surgery or injury. These methods may be carried out in various organs such as prostate, diaphragm, extremities, bladder, or bowel.

Another aspect of the disclosure provides a kit comprising
a composition comprising a DUOC-01 cell product in a pharmaceutically acceptable carrier, wherein DUOC-01 cell product comprises cells derived from cord blood mononuclear cells, wherein such cells express one or more of CD45, CD11b, CD14, CD16, CD206, CD163, Iba1, HLA-DR, TREM 2, and iNOS macrophage or microglia markers; and wherein such cells secrete IL-6, IL-10, or both; and
a label or instructions for administration of the composition to treat demyelinating condition.

In certain embodiments of the disclosure, the kits include a label or instruction to treat multiple sclerosis. In certain embodiments of the disclosure, the kits include a label or instruction to treat leukodystrophies. In certain embodiments of the disclosure, the kits include a label or instruction to treat spinal cord injury.

Still another aspect of the present disclosure provides methods for treating demyelinating conditions by administering to a subject in need thereof a therapeutically effective amount of a DUOC-HC composition comprising a DUOC-01 cell product formulated in hydrocortisone (HC), wherein the DUOC-01 cell product comprises cells derived from cord blood mononuclear cells, wherein such cells express one or more of CD45, CD11b, CD14, CD16, CD206, CD163, Iba1, HLA-DR, TREM 2, and iNOS macrophage or microglia markers; and wherein such cells secrete IL-6, IL-10, or both.

In certain embodiments, the DUOC-01 cells are incubated in Ringer's lactate solution with hydrocortisone, thereby obtaining a DUOC-HC composition.

Still another aspect of the disclosure provides a kit comprising a DUOC-HC composition comprising a DUOC-01 cell product formulated in hydrocortisone (HC), wherein DUOC-01 cell product comprises cells derived from cord blood mononuclear cells, wherein such cells express one or more of CD45, CD11b, CD14, CD16, CD206, CD163, Iba1, HLA-DR, TREM 2, and iNOS macrophage or microglia markers; and wherein such cells secrete IL-6, IL-10, or both; and a label or instructions for administration of the DUOC-HC composition to treat a demyelinating condition.

### BRIEF DESCRIPTION OF THE DRAWINGS

The patent or application file contains at least one drawing executed in color. Copies of this patent or patent application publication with color drawing(s) will be provided by the Office upon request and payment of necessary fee.

The accompanying drawings are included to provide a further understanding of the methods and compositions of the disclosure, and are incorporated in and constitute a part of this specification. The drawings illustrate one or more embodiment(s) of the disclosure, and together with the description serve to explain the principles and operation of the disclosure.

FIGS. 1A-1D illustrate severe demyelination of the midline corpus callosum (CC) area and glial infiltration of NSG mouse brain by cuprizone feeding. FIG. 1A shows LFB-PAS staining of NSG mice brain after 5 weeks of feeding with (right panel) and without (left panel) 0.2% cuprizone (CPZ). The midline CC area is shown by dotted black boxes in the top panels and then shown at higher magnification in the lower panels. Myelinated axons in the CC of mice fed normal laboratory chow are stained blue. Demyelination of the midline CC region of CPZ-treated animals is shown by the absence of the black-colored fibers. Scale bars: 2,000 μm (×20 magnification) and 100 μm (×400 magnification). FIG. 1B shows myelin basic protein immunostaining (green) after 5 weeks of feeding without (left panel) and with CPZ (right panel). Two different magnifications (top row is ×100 and bottom row is ×400) of the CC areas are shown. CC areas are shown by white dotted lines. FIG. 1C shows immunostaining with microglial marker Iba1 (upper panels) and astrocyte marker GFAP (lower panels) after 5 weeks of feeding without (left panels) and with CPZ (right panels). CC areas are shown by white dotted lines. Scale bars: 200 μm. FIG. 1D shows quantitative analysis of area covered by Iba1-positive (upper panel) and GFAP-positive (lower panel) cells, indicative of their numbers, along the CC. Both Iba1-positive and GFAP-positive cell numbers were significantly higher in the CPZ-treated animals. *P<0.02, **P<0.004. n=3 mice per group. C, control. Data are presented as the mean±SEM.

FIGS. 2A-2D illustrate that DUOC-01 cells disseminated from the injection site and persisted in the brain for up to 1 week after intracranial injection. Cuprizone-fed (CPZ-fed) mice were stereotactically injected with CFSE-labeled DUOC-01 cells. All cell nuclei were stained with DAPI. FIG. 2A shows CFSE-labeled (white) DUOC-01 cells were found in numerous parts of the brain including the injection site. Scale bars: 200 μm. CC, corpus callosum; SV, subventricular. FIG. 2B shows representative images of CFSE-positive and human nuclei (HuN)-positive cells in the brain at the injection site 4 days after injection. Upper left panel is CFSE channel only, lower left panel is HuN channel only, right panel is merge of CFSE, HuN, and DAPI channels. FIG. 2C shows presence of DUOC-01 cells 7 days after injection at the CC. Upper left panel is CFSE channel only, lower left panel is HuN channel only, right panel is merge of CFSE, HuN, and DAPI channels. FIG. 2D shows presence of DUOC-01 cells deep (white arrow) into the brain parenchyma. Upper left panel is CFSE channel only, lower left panel is HuN channel only, right panel is merge of CFSE, HuN, and DAPI channels. Scale bars (FIG. 2B-2D): 100 μm.

FIGS. 3A-3B illustrate LFB-PAS staining analysis of effect of DUOC-01 treatment on remyelination following cessation of cuprizone (CPZ) treatment. FIG. 3A shows LFB-PAS staining 1 week after intracranial injection of CD14⁺ monocytes (lower panels), DUOC-1 cells (middle panels), or Ringer's solution (upper panels) in CPZ-fed NSG mice. Midline corpus callosum (CC) area is shown by dotted gray box. Scale bars: 2,000 μm (×20 magnification) and 100 μm (×400 magnification). FIG. 3B shows myelination score based on LFB-PAS staining of mice fed normal chow (control) or CPZ for 5 weeks 1 week after treatment of CPZ-treated mice with CD14⁺ monocytes, DUOC-01 cells, or Ringer's. DUOC-01 treatment for 1 week significantly increased the myelination in the CC area compared to Ringer's-injected controls. **P<5.962×10⁻⁵ for this study. The CD14⁺ cell-treated sample showed an increased amount of remyelination compared to the Ringer's-treated group, but it was significantly less than the DUOC-01-treated group. *P<0.003875. Data are presented as the mean±SEM. Statistical comparisons were performed using the Wilcoxon rank-sum test for clustered data using the clusrank package in R.

FIGS. 4A-4C illustrate immunostaining analysis of the effect of DUOC-01 treatment on remyelination following cessation of cuprizone (CPZ) treatment. In all images, myelin basic protein (MBP) staining is shown. Representative ×400 laser confocal images of sections of the corpus callosum (CC) area of CPZ-fed mice immunostained with antibodies against MBP and neurofilament-H (NFH panel) are shown 1 week after treatment with Ringer's solution (FIG. 4A), with DUOC-01 (FIG. 4B), or with CD14⁺ (FIG. 4C). Upper left panels show MBP (green channel), lower left panels show NFH, and right panels show enlarged merge images of MBP and NFH channels. Scale bars: 100 μm.

FIG. 5 illustrates electron microscopic analysis of remyelination status upon DUOC-01 treatment. Representative ×2,650 (upper panels) and ×8,800 (lower panels) electron micrographs of corpus callosum region of cuprizone-fed mice 1 week after injection of Ringer's solution (left panels) or DUOC-01 cells (right panels). Arrows indicate unmyelinated axons. Solid triangles indicate mitochondria; enlarged mitochondria are clearly visible in the Ringer's-treated group. Scale bars: 2.0 μm.

FIGS. 6A-6E illustrates morphometric analysis of electron micrographs of corpus callosum regions of DUOC-01- and Ringer's-treated mice. FIG. 6A shows number of myelinated axons present per ×8,800 electron microscopy field. Data are presented as the mean±SEM showing all the data points. *P<4.29×10⁻⁹. FIG. 6B shows average number of turns of myelin sheath around axons, with right panels showing a representative electron micrograph of the myelin turns in an axon. *P<3.4×10′. Scale bars: 100 nm. FIG. 6C shows scatter plot of g-ratios, showing axonal measurements from 3 different animals in each group. Horizontal lines indicate mean g-ratios. P<0.014. FIG. 6D shows average size of mitochondria (area in nm²). Mean difference is significant between DUOC-01 and Ringer's groups. *P≤9.3×10⁻⁵. FIG. 6E shows average number of mitochondria per ×8,800 field. The mean difference is significant between DUOC-01 and Ringer's groups. *P≤0.02. Each column represents the value of measurements from 3 different animals. Error bars indicate the SEM. Statistical comparisons were performed using an unpaired 2-tailed Student's t test.

FIGS. 7A-7C illustrate DUOC-01 cell treatment reduces severe astrogliosis and microglial infiltration. FIG. 7A shows a quantitative cellularity scoring of LFB-stained brain slices on a scale of 0 to 3. **P≤7.618×10⁻⁵, n≥5. Control, not cuprizone fed; CPZ, cuprizone fed; Ringer's, 1 week after Ringer's injection; DUOC-01, 1 week after DUOC-01 injection. Data are presented as the mean±SEM showing each data point. Statistical comparisons were performed using the Wilcoxon rank-sum test for clustered data using the clusrank package in R.

FIG. 7B shows cellularity status by immunostaining using astrocyte-specific (GFAP, right panels) and microglia-specific (Iba1, left panels) markers. Midline corpus callosum (CC) areas are shown in dotted line. Scale bars: 100 FIG. 7C shows quantitative analysis of area covered by Iba1-positive (upper panel) and GFAP-positive (lower panel) cells, indicative of their numbers, along the CC. Both the numbers of Iba1-positive (microglia) and GFAP-positive (astrocytes) cells were significantly lower in the DUOC-01-treated mice. *P<0.002; **P<0.01. n=3 mice per group. Areas covered by each channel (either GFAP or Iba1) per microscopic field were quantified by ImageJ software. Data are presented as the mean±SEM. Statistical comparisons were performed using an unpaired 2-tailed Student's t test.

FIGS. 8A-8B illustrate DUOC-01 treatment promotes oligodendrocyte proliferation. FIG. 8A shows representative image of corpus callosum area of brains of cuprizone-fed mice treated with DUOC-01 cells (lower panels) or Ringer's solution (upper panels) stained with antibodies against Olig2 and Ki67. Yellow arrows indicate nuclei positive for both Olig2 and Ki67, blue arrows indicate only Ki67-positive nuclei. Scale bars: 50 FIG. 8B shows average number of Olig2⁺ Ki67⁺ cells (indicating proliferating oligodendrocytes) present per ×400 microscopic field were significantly higher in DUOC-01-treated samples compared to the Ringer's control. *P<0.01. Statistical comparisons were performed using an unpaired 2-tailed Student's t test.

FIGS. 9A-9C illustrate comparative whole-transcriptome analysis of CD14 and DUOC-01 cells. FIG. 9A shows Venn diagram displaying the findings from microarray analysis showing the number of genes differentially expressed in purified fresh CD14⁺ (n=4) and DUOC-01 (n=3) cells as well as genes expressed by both cell types. MASS-normalized data were used to filter out expressed/nonexpressed genes. This figure represents the most stringent analysis; to be scored as expressed, the transcript had to be detected above background in all samples of a given cell type analyzed. See the text for expression figures at different stringencies. FIG. 9B shows Volcano plot depiction of findings from microarray analysis showing the genes differentially expressed in purified fresh CD14⁺ and DUOC-01 cells. The log₁₀ of Bonferroni-Hochberg-corrected P values in ANOVA (y axis) is plotted against the of fold change between 2 groups (x axis). Red lines delineate the cutoffs for genes significantly (P<0.05) downregulated (left) or upregulated (right) in DUOC-01 cells. Each data point represents 1 gene probe set. FIG. 9C shows heat maps showing differentially expressed genes. Up- and downregulated genes are displayed in red and blue, respectively. There were 9,645 genes that are differentially expressed at a magnitude of at least 2-fold.

FIG. 10 illustrates a diagram of experimental design for manufacture of DUOC-01 cell product. GM and NTM are the growth medium and neurotrophic medium described in Materials and Methods. Day 0 cells were not cultured. Day 14 samples were from cells that were cultured in GM only and had not been exposed to NTM medium. Day 21 cells were cultured in 50% NTM medium/50% GM for 3 days and then 25% NTM/75% GM medium for 4 days.

FIG. 11 illustrates the changes in expression of selected transcripts during manufacture of cell products from cord blood CD14+ monocytes and cord blood mononuclear cells. Cultures were initiated with either cell population as described in the text, and cell products were harvested on the days shown and analyzed by qPCR for expression of the genes indicated. Each time point shows the mean±SEM ΔCq value normalized to glyceraldehyde 3-phosphate dehydrogenase (GAPDH) for experiments done with three CB units. Increase in ΔCq indicates a decrease in transcript abundance, and decrease indicates an increase in abundance relative to GAPDH.

FIG. 12 illustrates changes in expression of 77 genes during manufacturing of cell products from CB MNC or CB CD14+ monocytes. Cultures were initiated with CB MNC (dark gray points) or CB CD14+ monocytes (light gray points). Cell products were harvested after 14 days (left column of data for each gene) or 21 days (right column of data) and analyzed by qPCR for expression of the genes indicated on the abscissa. Data points for both cell populations derived for each of three CB units are shown; some of these six points overlap in this format. The ordinate units are ΔΔCt value normalized to glyceraldehyde 3-phosphate dehydrogenase (GAPDH) expression in each sample and to expression by freshly isolated CD14+ monocytes from the CB unit used to start the culture. Thus, positive values indicate that transcript is overexpressed in freshly isolated CD14+ monocytes, and negative values mean that the transcript is over-expressed in the cultured cell population. The ordinate values are powers of 2; the line on each panel shows ΔΔCt=0, or no change in expression relative to freshly isolated CD14+ monocytes.

FIG. 13 Illustrates concentration of chemokines, cytokines and metal metalloproteases accumulating in culture supernatants during manufacturing. Ordinate: picogram/milliliter measured by Bioplex; note that scales are different for each set of proteins. Abscissa: days in culture; for each protein, results for 14-day supernatants are plotted on the left, and 21-day supernatants, on the right. Data from three cords are shown; each point is the mean value for three analyses performed on an individual cell product. Diamonds are data from cultures initiated with purified CB CD14+ monocytes. Circles are data from cultures initiated with CB mononuclear cells from the same cords. In the standard protocol for manufacturing DUOC-01, CB mononuclear cells are cultured for 21 days.

FIG. 14 illustrates viability of DUOC-01 cells over time when incubated with hydrocortisone (HC) compared to DUOC-01 cells incubated with dexamethasone (Dex) or Ringer's solution. Viability was measured by AOPI using the Nexcelom Cellometer Auto 2000 (n=6 samples per time point; 2-way ANOVA p<0.001 for treatment factor; Tukey's post-hoc ***>0.001).

FIG. 15 illustrates expression of CD45, CD11b, Trem2, and CD14 analyzed by flow cytometry (n=6 per condition). DUOC-01 cells were collected 4 hours after adding HC or Dex.

FIG. 16 illustrates myelination scores of murine cuprizone model of demyelination one week after treatment with Ringer's, Ringer's+HC, DUOC-01 cells, or DUOC-01 cells exposed to HC (DUOC-HC) (1×10⁵ cells injected). Data are presented as mean±SEM (ANOVA for cells P<0.001; n=9-16 mice per group, * P<0.05, Tukey's post-hoc test; pooled 2 independent experiments).

FIG. 17 illustrates average clinical scores of experimental autoimmune encephalomyelitis (EAE) mice injected with Ringer's, Ringer's+HC, DUOC-01 in Ringer's, or DUOC-01 incubated in HC (DUOC-HC) (n=17-48 mice per group; mixed-effects model p<0.0001 for time and treatment. Tukey post-hoc * p<0.05 and ** p<0.01). Data are presented as mean±SEM. Red arrow delineates the injection Day 0.

### DETAILED DESCRIPTION OF THE INVENTION

Before the disclosed processes and materials are described, it is to be understood that the aspects described herein are not limited to specific embodiments, apparati, or configurations, and as such can, of course, vary. It is also to be understood that the terminology used herein is for the purpose of describing particular aspects only and, unless specifically defined herein, is not intended to be limiting.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used in the specification and the appended claims, the singular forms “a,” “an” and “the” include plural referents unless the context clearly dictates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the term “contacting” includes the physical contact of at least one substance to another substance.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

The term “effective amount” or “therapeutically effective amount” refers to an amount sufficient to effect beneficial or desirable biological and/or clinical results.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. Preferably, the subject is a human patient that is at for, or suffering from, multiple sclerosis.

As used herein, the term “disease” refers to any condition that is abnormal, such as a disorder or a structure or function, that affects part or all of a subject.

As used herein, the term “multiple sclerosis” refers to a neurological disorder that involves the degradation and/or destruction and/or deterioration of the myelin sheath.

In view of the present disclosure, the methods and compositions described herein can be configured by the person of ordinary skill in the art to meet the desired need. In general, the disclosed materials, methods, and apparati provide improvements in treatment of demyelinating conditions, particularly those that do not arise from enzyme deficiency. The inventors found that DUOC-01 cell product accelerates brain remyelination after cuprizone-induced (CPZ-induced) demyelination. CPZ-induced demyelination model has been widely used to study the mechanisms and cellular dynamics of remyelination in the corpus callosum (CC) region. CPZ is a Cu⁺⁺-chelating agent that is highly toxic to oligodendrocytes, and CPZ feeding results in demyelination that can be assessed in the CC where abundant neural fiber bundles become disorganized as myelin degrades. When CPZ is removed from the diet, newly differentiated oligodendrocytes remyelinate the CC over a period of weeks. Astrocytes, microglia, and infiltrating peripheral monocytes have been shown to participate in the remyelination process in this model. CPZ feeding in immunodeficient NOD/SCID/IL2Rγ^{null} (NSG) mice (i.e., mice that lack functional T cells, B cells, and NK cells and readily accept human tissue grafts) results in reversible demyelination in the CC with a time course similar to the process in immune-competent mouse strains. This model was used to assess the activity of DUOC-01 cell product in promoting brain remyelination.

The inventors also found that uncultured CD14⁺ CB cells that give rise to DUOC-01 also accelerate remyelination, but significantly less actively than DUOC-01 cells. A comparison of whole-genome expression arrays of CB CD14⁺ monocytes and DUOC-01 revealed large differences in gene expression, and helped identify candidate molecules that may participate in remyelination. The cells in the DUOC-01 product express and secrete several factors that promote myelination by several mechanisms.

Thus, one aspect of the disclosure provides methods for treating demyelinating conditions, such as leukodystrophies, multiple sclerosis, or spinal cord injury. Such methods include administering to the subject in need thereof a therapeutically effective amount of a composition comprising a DUOC-01 cell product in a pharmaceutically acceptable carrier.

In certain embodiments of the disclosure, the methods are for treating multiple sclerosis in a subject. In certain embodiments of the disclosure, the methods are for treating leukodystrophies in a subject. In certain embodiments of the disclosure, the methods are for treating spinal cord injury in a subject.

As noted above, the compositions useful in the methods of the disclosure include a DUOC-01 cell product. These cells were described by Kurtzberg J, et al. (Cytotherapy. 2015; 17(6):803-815), Saha A, et al. (JCI Insight. 2016; 1(13):e86667), and Scotland P (Cytotherapy. 2017; 19(6):771-782), all incorporated by reference in their entirety. As understood by those in the art, the DUOC-01 cell product includes cells derived from cord blood mononuclear cells. In certain embodiments, such cells express one or more (e.g., one, two, three, four, or more) of CD45, CD11b, CD14, CD16, CD206, CD163, Iba1, HLA-DR, TREM 2, and iNOS macrophage or microglia markers. In certain embodiments, at least 50% of the cell population, e.g., at least 60%, or at least 70%, or at least 80%, or at least 85%, or even at least 90% of the cell population, expresses one or more (e.g., one, two, three, four, or more) of CD45, CD11b, CD14, CD16, CD206, CD163, Iba1, HLA-DR, and iNOS macrophage or microglia markers.

In certain embodiments, the DUOC-01 cell product includes cells that secrete IL-6, IL-10, or both. In certain embodiments, the concentration of IL-6 in DUOC-01 cell product is between about 300 to about 2600 pg/10⁶ cells/mL. In certain embodiments, the concentration of IL-10 in DUOC-01 cell product is between about 20 to about 250 pg/10⁶ cells/mL.

In certain embodiments, the DUOC-01 cell product includes cells that overexpress one or more of platelet-derived growth factor subunit A (PDGFA), KIT-ligand (KITLG, also known as stem cell factor [SCF]), insulin-like growth factor-1 (IGF1), triggering receptor expressed on myeloid cells 2 (TREM2), matrix metalloproteinase-9 (MMP9), and MMP12 transcripts. In certain embodiments, the expression of one or more of PDGFA, KITLG, IGF1, TREM2, MMP9, and MMP12 transcripts is at least 5 times higher compared to CB CD14⁺ monocytes, e.g., at least 10 times higher, or at least 15 times higher, or at least 20 times higher, or at least 25 times higher, or at least 30 times higher, or at least 50 times higher, or at least 100 times higher, or even 1000 times higher.

In certain embodiments, the DUOC-01 cell product includes cells that have unique RNA expression profile relative to CB CD14⁺ monocytes. For example, in certain embodiments, the RNA expression profile is as set forth in Table 2.

In certain embodiments, the DUOC-01 cell product excludes cells expressing CD3 (i.e., DUOC-01 cell product cells do not express CD3). In certain embodiments, no more than 1% of the cell population, e.g., or no more than 0.5%, or no more than 0.1%, or even 0% of the cell population, expresses CD3 marker.

In certain embodiments, the DUOC-01 cell product may be a partially human leukocyte antigen (HLA)-matched to the subject.

The route of administration of the compositions of the disclosure may be selected by one of skill in the art based on the diseases treated and desired results. Thus, in one embodiment, the composition is administered via local tissue injection, intrathecally (e.g., an administration into the spinal canal, or into the subarachnoid space, or into space under the arachnoid membrane of the brain), or intracerebrally (e.g., an administration into the cerebrum). In certain embodiments, the composition is administered intrathecally, such as via an intrathecal injection. In certain embodiments, the composition is administered via local tissue injection, e.g., into a local area where a peripheral nerve has been damaged. For example, in certain embodiments, the local tissue injection may be into the tissue adjacent to the damaged nerve (e.g., prostate, diaphragm, extremities, bladder, bowel, etc.)

The compositions of the disclosure may be administered in a single dose. The compositions of the disclosure may also be administered in multiple doses (e.g., two, three, or more single doses per treatment) over a time period (e.g., minutes, hours, or even several days). In certain embodiments, the compositions of the disclosure may be administered over a time period in the range of about 1 second to about 3 minutes, e.g., over about 60 seconds to about 120 seconds, or over about 90 seconds to about 120 seconds, or over about 60 seconds to about 180 seconds, over about 90 seconds to about 180 seconds, or over about 1 seconds to about 15 seconds, or over about 5 seconds to about 15 seconds, or over about 1 seconds to about 30 seconds, or over about 5 seconds to about 30 seconds, or over about 15 seconds to about 60 seconds, or over about 15 seconds to about 90 seconds.

The DUOC-01 cell product may be present in the composition in a therapeutically effective concentration. In certain embodiments, the concentration of DUOC-01 cell product in the composition is about 1×10⁵ to about 1×10⁸ cells/dose of composition; e.g., about 1×10⁶ to about 1×10⁸ cells/dose, or about 1×10⁷ to about 1×10⁸ cells/dose, or about 1×10⁶ to about 5×10⁷ cells/dose, about 1×10⁵ to about 1×10⁷ cells/dose, or about 1×10⁶ to about 1×10⁷ cells/dose, or about 1×10⁶ to about 5×10⁶ cells/dose, or about 1×10⁶ to about 5×10⁶ cells/dose of composition. One of skill in the art will recognize that suitable volume of the dose may be selected based on the desired route of administration. For example, intrathecal administration may use dose volumes in the range of about 1 mL to about 10 mL; e.g., about 5 mL, or about 4 mL to about 6 mL, or about 3 mL to about 7 mL, or about 1 mL to about 5 mL, or about 5 mL to about 10 mL. For example, intracerebral administration or local tissue injection may use dose volumes in the range of about 0.5 mL to about 2 mL; e.g., about 1 mL, or about 0.5 mL to about 1.5 mL, or about 0.5 mL to about 1 mL, or about 1 mL to about 1.5 mL, or about 5 mL to about 10 mL.

In certain embodiments, the DUOC-01 cell product is present in the composition in the amount of about 1×10⁵ to about 1×10⁸ cells; e.g., about 1×10⁵ to about 1×10⁷ cells, or about 1×10⁵ to about 1×10⁶ cells, or about 1×10⁶ to about 1×10⁸ cells, or about 1×10⁶ to about 1×10⁷ cells, or about 1×10⁶ to about 5×10⁶ cells.

Any suitable pharmaceutically acceptable carrier may be used in the compositions of the disclosure. In certain embodiments, the pharmaceutically acceptable carrier is Ringer's lactate solution. In certain embodiments, the pharmaceutically acceptable carrier is Ringer's solution, Tyrode's solution, or a saline solution.

The compositions useful in the methods of the disclosure may be obtained by exposing the cord blood mononuclear cells in a first culture medium to one or more factors selected from: platelet-derived growth factor (PDGF), neurotrophin-3 (NT-3), vascular endothelial growth factor (VEGF), and triiodothyronine (T₃); and at least one of serum or plasma for a period of time sufficient to obtain DUOC-01 cell product. After isolating the DUOC-01 cell product, the DUOC-01 cell product may be dissolved in the pharmaceutically acceptable carrier to obtain the composition of the disclosure. In certain embodiments, an additional amount of PDGF, NT-3, VEGF, T₃, and serum after 7 days and after 17 days may be provided. In certain embodiments, an additional amount of PDGF, NT-3, and VEGF after 14 days may be provided.

In certain embodiments, the period of time sufficient to obtain DUOC-01 cell product is about 21 days. In certain embodiments, the period of time sufficient to obtain DUOC-01 cell product is about 17 days, or 18 days, or 19 days, or 20 days, or 22 days, or 23 days, or 24 days.

In certain embodiments, the PDGF is present in a concentration of about 1 to about 10 ng/mL. In certain embodiments, the NT-3 is present in a concentration of about 0.1 to about 5 ng/mL. In certain embodiments, the VEGF is present in a concentration of about 1 to about 50 ng/mL. In certain embodiments, T₃ is present in a concentration of about 10 to about 100 ng/mL.

In certain embodiments, exposing the cord blood mononuclear cells in a first culture medium is to PDGF, NT-3, VEGF, T₃, and serum.

Another aspect of the disclosure provides a kit comprising a composition comprising a DUOC-01 cell product as described herein in a pharmaceutically acceptable carrier; and a label or instructions for administration of the composition to treat a demyelinating condition. In certain embodiments of the disclosure, the kit is for treating multiple sclerosis in a subject. In certain embodiments of the disclosure, the kit is for treating leukodystrophies in a subject. In certain embodiments of the disclosure, the kit is for treating spinal cord injury in a subject.

Still another aspect of the present disclosure provides methods for treating demyelinating conditions by administering to a subject in need thereof a therapeutically effective amount of a DUOC-HC composition comprising a DUOC-01 cell product formulated in hydrocortisone, wherein the DUOC-01 cell product comprises cells derived from cord blood mononuclear cells, wherein such cells express one or more of CD45, CD11b, CD14, CD16, CD206, CD163, Iba1, HLA-DR, TREM 2, and iNOS macrophage or microglia markers; and wherein such cells secrete IL-6, IL-10, or both.

In certain embodiments, the DUOC-01 cells are incubated in Ringer's lactate solution with hydrocortisone, thereby obtaining a DUOC-01 composition.

In certain embodiments, the demyelinating condition may include, but is not limited to, multiple sclerosis, leukodystrophy, spinal cord injury, peripheral nerve damage, Parkinson's disease, amyotrophic lateral sclerosis (ALS), or Alzheimer's disease.

In certain embodiments, the DUOC-01 cell product excludes cells expressing CD3.

For the treatment of a demyelinating condition, the composition may be administered in a single dose or in multiple doses via local tissue injection or intrathecally. In certain embodiments, the concentration of the DUOC-01 cell product in the composition is about 1×10⁵ to about 1×10⁸ cells/dose of composition; e.g., about 1×10⁶ to about 1×10⁸ cells/dose, or about 1×10⁷ to about 1×10⁸ cells/dose, or about 1×10⁶ to about 5×10⁷ cells/dose, about 1×10⁵ to about 1×10⁷ cells/dose, or about 1×10⁶ to about 1×10⁷ cells/dose, or about 1×10⁶ to about 5×10⁶ cells/dose, or about 1×10⁶ to about 5×10⁶ cells/dose of composition. One of skill in the art will recognize that suitable volume of the dose may be selected based on the desired route of administration. For example, intrathecal administration may use dose volumes in the range of about 1 mL to about 10 mL; e.g., about 5 mL, or about 4 mL to about 6 mL, or about 3 mL to about 7 mL, or about 1 mL to about 5 mL, or about 5 mL to about 10 mL. For example, intracerebral administration or local tissue injection may use dose volumes in the range of about 0.5 mL to about 2 mL; e.g., about 1 mL, or about 0.5 mL to about 1.5 mL, or about 0.5 mL to about 1 mL, or about 1 mL to about 1.5 mL, or about 5 mL to about 10 mL.

In certain embodiments, the DUOC-01 cell product is present in the composition in the amount of about 1×10⁵ to about 1×10⁸ cells; e.g., about 1×10⁵ to about 1×10⁷ cells, or about 1×10⁵ to about 1×10⁶ cells, or about 1×10⁶ to about 1×10⁸ cells, or about 1×10⁶ to about 1×10⁷ cells, or about 1×10⁶ to about 5×10⁶ cells.

The compositions useful in the methods disclosed above and herein may be obtained by exposing the cord blood mononuclear cells in a first culture medium to one or more factors selected from: platelet-derived growth factor (PDGF), neurotrophin-3 (NT-3), vascular endothelial growth factor (VEGF), and triiodothyronine (T₃); and at least one of serum or plasma for a period of time sufficient to obtain DUOC-01 cell product. After isolating the DUOC-01 cell product, the DUOC-01 cell product may be dissolved in a pharmaceutically acceptable carrier to obtain the composition of the disclosure. In certain embodiments, an additional amount of PDGF, NT-3, VEGF, T₃, and serum after 7 days and after 17 days may be provided. In certain embodiments, an additional amount of PDGF, NT-3, and VEGF after 14 days may be provided.

Any suitable pharmaceutically acceptable carrier may be used in the compositions of the disclosure. In certain embodiments, the pharmaceutically acceptable carrier is Ringer's lactate solution with hydrocortisone. In certain embodiments, the pharmaceutically acceptable carrier is Ringer's solution, Tyrode's solution, or a saline solution, with hydrocortisone.

In certain embodiments, the period of time sufficient to obtain DUOC-01 cell product is about 21 days. In certain embodiments, the period of time sufficient to obtain DUOC-01 cell product is about 17 days, or 18 days, or 19 days, or 20 days, or 22 days, or 23 days, or 24 days.

In certain embodiments, the PDGF is present in a concentration of about 1 to about 10 ng/mL. In certain embodiments, the NT-3 is present in a concentration of about 0.1 to about 5 ng/mL. In certain embodiments, the VEGF is present in a concentration of about 1 to about 50 ng/mL. In certain embodiments, T₃ is present in a concentration of about 10 to about 100 ng/mL.

In certain embodiments, exposing the cord blood mononuclear cells in a first culture medium is to PDGF, NT-3, VEGF, T₃, and serum.

Still another aspect of the disclosure provides a kit comprising a DUOC-HC composition comprising a DUOC-01 cell product formulated in hydrocortisone (HC), wherein DUOC-01 cell product comprises cells derived from cord blood mononuclear cells, wherein such cells express one or more of CD45, CD11b, CD14, CD16, CD206, CD163, Iba1, HLA-DR, TREM 2, and iNOS macrophage or microglia markers; and wherein such cells secrete IL-6, IL-10, or both; and a label or instructions for administration of the composition to treat a demyelinating condition.

In certain embodiments, the DUOC-01 cells overexpress one or more of PDGFA, KITLG/SCF, IGF1, TREM2, MMP9, and MMP12 transcripts.

In certain embodiments, the amount of the DUOC-HC composition is sufficient to provide about 1×10⁵ to about 1×10⁸ DUOC-01 cells. By way of non-limiting example, the amount of composition is sufficient to provide about 1×10⁶ to about 1×10⁸ cells/dose, or about 1×10⁷ to about 1×10⁸ cells/dose, or about 1×10⁶ to about 5×10⁷ cells/dose, about 1×10⁵ to about 1×10⁷ cells/dose, or about 1×10⁶ to about 1×10⁷ cells/dose, or about 1×10⁶ to about 5×10⁶ cells/dose, or about 1×10⁶ to about 5×10⁶ cells/dose of composition.

The compositions comprised in the kits disclosed above and herein may be obtained by exposing the cord blood mononuclear cells in a first culture medium to one or more factors selected from: platelet-derived growth factor (PDGF), neurotrophin-3 (NT-3), vascular endothelial growth factor (VEGF), and triiodothyronine (T₃); and at least one of serum or plasma for a period of time sufficient to obtain DUOC-01 cell product. After isolating the DUOC-01 cell product, the DUOC-01 cell product may be dissolved in a pharmaceutically acceptable carrier to obtain the DUOC-HC composition of the disclosure.

Any suitable pharmaceutically acceptable carrier may be used in the compositions of the disclosure. In certain embodiments, the pharmaceutically acceptable carrier is Ringer's lactate solution with hydrocortisone. In certain embodiments, the pharmaceutically acceptable carrier is Ringer's solution, Tyrode's solution, or a saline solution, with hydrocortisone.

In certain embodiments, the kits disclosed above and herein may be used to treat a demyelinating condition, including but not limited to, multiple sclerosis, leukodystrophy, peripheral nerve disease, spinal cord injury, Parkinson's disease, amyotrophic lateral sclerosis (ALS), or Alzheimer's disease

Certain aspects of the disclosure are now explained further via the following non-limiting examples.

### EXAMPLES

Materials and Methods

Manufacture of DUOC-01:

The umbilical cord blood (UCB) cell suspension was washed with dextran (Hospira, Lake Forest, Ill.)/albumin (Grifols, Los Angeles, Calif.) wash using the Sepax Cell Processing System's Cord Wash program (Biosafe), manual processing, or using the SynGenX-Lab instrument. The UCB cell suspension was then removed from the product bag and diluted in 450 mL of PBS (Life Technologies, Carlsbad, Calif.) supplemented with 1% human serum albumin (HSA) and 0.4 μL/mL (100 units/mL) benzonase nuclease (EMD Millipore, Burlington, Mass.). Cells were centrifuged and suspended in a smaller volume of PBS/HSA. Mature erythrocytes are removed using an antibody to CD235a (Glycophorin-A) and magnetic nanoparticles (EasySep™ Human Glycophorin A Depletion Kit, Stem Cell Technologies, Vancouver, Canada). The resulting cell population was suspended in Oligodendrocyte medium (α-MEM (Life Technologies, Carlsbad, Calif.) supplemented with 10% fetal calf serum (Life Technologies, Carlsbad, Calif.), insulin-transferrin-selenium (Invitrogen, Carlsbad, Calif.), 5 ng/mL platelet derived growth factor (PDGF) (Peprotech, Rocky Hill, N.J.), 1 ng/mL neurotrophin-3 (NT-3) (Peprotech, Rocky Hill, N.J.), 10 ng/mL vascular endothelial growth factor (VEGF) (Peprotech, Rocky Hill, N.J.), 30 ng/mL triiodothyronine (Sigma-Aldrich, St. Louis, Mo.) and plated in sterile tissue culture flasks at a concentration of 5×10⁵ cells/cm². The flasks were then incubated at 35-37° C./5% CO₂ for 21 days. On day 7 of culture, half or all of the medium was removed and replaced with an equal volume of fresh Oligodendrocyte medium. On day 14 of culture, half the volume of medium was exchanged for an equal volume of neurotrophic medium containing Neurocult NS-A basal medium (Stem Cell Technologies, Vancouver, Canada), Neurocult NS-A differentiation supplement (Stem Cell Technologies, Vancouver, Canada), and PDGF, VEGF, and NT-3 in the concentrations listed above for Oligodendrocyte medium. On day 17 of culture, half the volume of medium was exchanged for an equal volume of Oligodendrocyte medium (supplemented alpha-MEM). On day 19 of culture, one flask was harvested for initial sterility testing and characterization of the cellular content by immunophenotyping. Supplemental feeding was given if robust growth of cells was observed. On day 19-21 of culture, the remaining flasks are harvested, release and mycoplasma testing was performed, and the DUOC-01 product was formulated in its final excipient (e.g., Lactated Ringers solution) and container/closure system at the appropriate dosage for the recipient's study cohort. If the lot of DUOC-01 failed to meet release specifications, it was not administered.

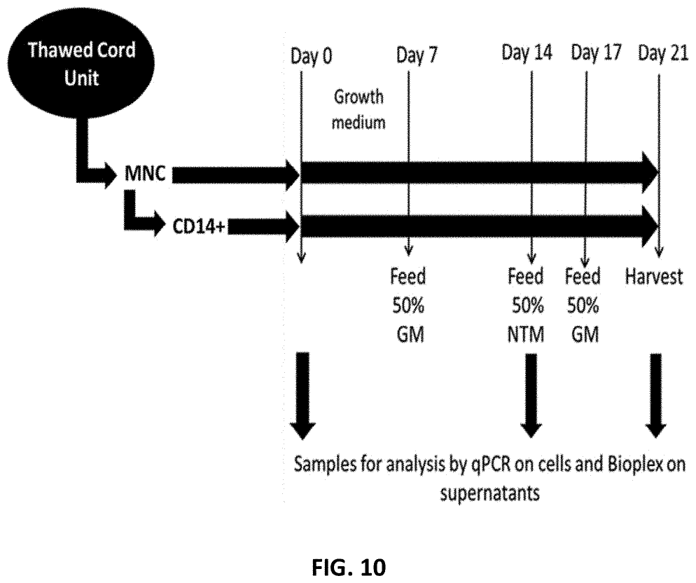

In some embodiments, the strategy for manufacturing of call product is illustrated in FIG. 10.

Separation of Specific Cell Populations from CB:

CD14⁺ populations from cryopreserved CB were immunomagnetically selected using Whole Blood CD14 Microbeads as described by the manufacturer (Miltenyi Biotec). Cells that did not adhere to the anti-CD14 antibody columns comprised the CD14⁺-depleted population. Some experiments were carried out with cells from CD14⁺ cells from freshly collected CB. MNC populations depleted of erythrocytes were prepared from fresh CB either by centrifugation on Ficoll or in SepMate tubes (STEMCELL Technologies) as described by the manufacturer. CD14⁺ cells were immunomagnetically purified from MNC preparations using the CD14 Microbeads. Similar experiments were carried out with CB cell populations enriched for or depleted of CD34-expressing cells using anti-CD34 Microbeads (Miltenyi Biotec).

To prepare CD14+ cell RNA for microarray analysis, freshly collected CB was centrifuged on Ficoll to prepare MNC fractions. These fractions were treated with 0.15 M NH₄Cl to lyse erythrocytes, washed in PBS, and then incubated on ice with PeCy7-mouse anti-human CD14, FITC-mouse anti-human CD3, and FITC-mouse anti-human CD235a antibodies (all from BD, San Jose, Calif.). Cells were then sorted twice by flow cytometry to yield CD14+CD235a−CD3− populations. The first enrichment sort was followed by a second purity sort. Cells were maintained at 0° C.-4° C. during all procedures, including flow sorting. The purity of selected populations and the extent of CD14+ cell depletion were determined by flow cytometry as previously described (Kurtzberg J, et al. Cytotherapy. 2015; 17(6):803-815.)

CPZ Demyelination in NSG Mice:

Eight-week-old male NSG mice were acclimated to milled standard rodent chow for 1 week. Demyelination was subsequently induced by incorporating 0.2% by weight CPZ (bis-cyclohexanone oxaldihydrazone, Sigma-Aldrich) into the milled chow for 5 weeks. Brains were then harvested from CPZ-fed animals and controls were fed chow without CPZ for subsequent assessment of the degree of demyelination and disruption of brain histology induced by CPZ. To assess the effects of cell treatment, 2 additional groups of animals were returned to standard diet to allow remyelination. One day after the change in diet, animals were stereotactically injected in the CC (coordinates: 0.2 mm posterior and 1.1 mm lateral to the bregma, and 1.5 mm deep from the skull surface) with 10⁵ cells (DUOC-01 or CD14⁺) in 5 μl of lactated Ringer's solution or with excipient within the 2-hour expiry period for the DUOC-01 clinical cell product. One week following treatment, brains were harvested by intracardiac perfusion with PBS and then with 4% paraformaldehyde. Paraffin-embedded coronal sections were prepared for analysis of myelination status, the organization of neural fibers, and persistence of injected human cells by LFB-PAS staining, immunohistochemistry, and electron microscopy as described below. Cohorts of 5 or 6 mice were analyzed under each set of experimental conditions.

Myelination, cellular infiltration, and gliosis were assessed by LFB-PAS staining of the CC region, (approximately at the level of the bregma −0.2 to −0.9 mm) (Doan V, et al. J Neurosci Res. 2013; 91(3):363-373). 5.0-μm-thick paraffin-embedded coronal sections of the CC region were used. LFB stains the myelin blue, and PAS stains demyelinated axons pink. Three independent, blinded readers scored coded LFB-PAS-stained sections between 0 and 3. A score of 3 is equivalent to the myelin status of a brain not treated with CPZ; 0 is equivalent to a completely demyelinated brain area. A score of 1 or 2 corresponds to one-third or two-third fiber myelination, respectively. Similarly, a quantitative cellularity score was obtained by counting the number of nuclei in the CC region of LFB-stained brain slices on a scale of 0 to 3, by blinded readers.

Immunohistochemistry:

Brain slices from 3 animals in each treatment group were analyzed. Primary antibodies used were: rat anti-MBP (1:1,000, Abcam, Cambridge, United Kingdom); chicken anti-NFH (1:100,000, EnCor Biotech, Gainesville, Fla.); mouse anti-HuN (1:250, Millipore, Burlington, Mass.); chicken anti-GFAP (1:500, Abcam); goat anti-Iba1 (1:200, Abcam); rabbit anti-Ki67 (1:300, Abcam); and goat anti-Olig2 (1:50, R&D Systems, Minneapolis, Minn.). Secondary antibodies used were: Alexa-488 donkey anti-rat, Alexa-647 donkey anti-chicken, Alexa-568 donkey anti-mouse (1:500, Molecular Probes, Eugene, Oreg.). Confocal micrographs were obtained using constant settings including laser power, stack thickness, and camera resolution. The number of stained cells per microscopic field in the CC region and the average area covered by cells stained with each antibody were quantified by ImageJ software (NIH).

Electron Microscopy:

Brains were prepared for electron microscopy. Images were then analyzed using ImageJ software. For analysis, g-ratio analysis was modified such that the inner diameter of compact myelin (instead of the axon diameter) was divided by the outer diameter of the myelin sheath. Diameters were calculated from enclosed areas. Fibers with prominent outfoldings in the plane of section were excluded. A plugin for the ImageJ software (http://rsbweb.nih.gov/ij) was implemented, which allowed for semiautomated analysis of randomly selected sets of fibers (Goebbels S, et al. J Neurosci. 2010; 30(26):8953-8964.). Plugin and source code are available online (http://gratio.efil.de). A minimum of 100 fibers/mouse, 3 mice/time point/treatment, were analyzed. The number of mitochondria in all cells in the CC area was counted in all the electron micrographs, and average mitochondria present per ×8,800-magnified field was calculated. To determine the size of the mitochondria, electron microscopic images were analyzed with ImageJ, using the area analysis function. For area measurement, the mitochondria were circled by the lasso tool, and then the areas of the circles were calculated and converted to their actual values using the scale bar. At least 10 images were analyzed per sample in a blinded fashion.

Tracking DUOC-01 Cells in the Brain:

DUOC-01 cells were stained with 5 μM Vybrant CFDA SE Cell Tracer dye (CFSE, V12883, green fluorescence, Life Technologies) and injected into the CC as described above. One, four, and seven days later, brains were harvested, sliced, and processed for confocal microscopy.

Expression Analysis by Microarrays:

RNA for microarray analysis was prepared from 4 flow-sorted CD14⁺ CB and 3 DUOC-01 products using the QIAGEN RNeasy Mini Kit as described by the manufacturer. These samples were used for whole-genome microarray analysis on 1 chip. Microarray analysis was performed by the Microarray Shared Resource in the Duke Center for Genomic and Computational Biology using Affymetrix GeneChip Human Transcriptome Array 2.0 microarrays. Partek Genomics Suite 6.6 (Partek Inc.) was used to perform data analysis. Robust multichip analysis (RMA) normalization was performed on the entire dataset. Multi-way ANOVA and analysis of the fold change were performed to select target genes that were differentially expressed. Hierarchical clustering was performed on differentially expressed genes based on average linkage with Pearson's dissimilarity.

RNA Isolation and Quantitative Real-Time PCR:

Quantitative real-time RT-PCR was used to measure levels of transcripts in CD14⁺ CB cells, DUOC-01, and cell products manufactured from isolated CD14⁺ CB cells using the RNeasy Mini Kit with DNAse-1 treatment as instructed. The cDNA was synthesized from equal amounts of RNA using SuperScript III enzyme, oligo(dT) primers, dNTPs, RNase Out, DTT, and buffer as instructed (Life Technologies). Diluted cDNA was amplified on the Bio-Rad CFX96 Real Time System using SsoAdvanced Universal SYBR Green Supermix (Bio-Rad) and the following oligonucleotides: PDGFA (sense 5′-CTTCCTCGATGCTTCTCTTCC-3′, antisense 5′-GACCTCCAGCGACTCCT-3′); MMP9 (sense 5′-TGTACCGCTATGGTTACACTCG-3′, antisense 5′-GGCAGGGACAGTTGCTTCT-3′); IGF1 (sense 5′-GCCTCCTTAGATCACAGCTC-3′, antisense 5′-GATGCTCTTCAGTTCGTGTGT-3′); IL10 (sense 5′-GCGCTGTCATCGATTTCTTC-3′, antisense 5′-TCACTCATGGCTTTGTAGATGC-3′); MMP12 (sense 5′-CAAAACTCAAATTGGGGTCACAG-3′, antisense 5′-CTCTCTGCTGATGACATACGTG-3′), KITLG (sense 5′-AGCTGAAGATAAATGCAAGTGAG-3′, antisense 5′-CAGAACAGCTAAACGGAGTCG-3′), and TREM2 (sense 5′-TCATAGGGGCAAGACACCT-3′, antisense 5′-GCTGCTCATCTTACTCTTTGTC-3′). Values were normalized to GAPDH expression.

Accumulation of Human Proteins in Culture Medium:

The concentrations of 16 secreted proteins in supernatants removed from cultures initiated with CB MNC or with purified CD14+ monocytes derived from the same cord were compared. Supernatants were collected before feeding on day 14 and during harvesting of final cell products on day 21. Secreted protein concentrations were measured [8] by antibody capture immunoassay with fluorescence reporters using the Bio Rad Bioplex 200 instrument. IL-6, IL-10 and 10 chemokines were multiplexed on one plate (Biorad catalog no. 171-AK99MR2, standard lot no. 5036571), four human matrix metalloproteases on a second (catalog no. 171-AM001M, standard lot no. 5042979, and MIP-1β was assayed on the third (catalog no. 171-D50001, standard lot no. 5039890). Standards for each protein provided by the manufacturer were diluted in appropriate uninoculated tissue culture medium to construct standard curves, and the concentration of each protein in the supernatants was calculated.

Statistics:

In most cases statistical comparisons were conducted with 2-tailed Student's t tests with unequal variance. For comparing LFB and cellularity scores, Wilcoxon rank-sum tests were used. Statistical comparisons were performed using the Wilcoxon rank-sum test for clustered data using the clusrank package in R. Mean differences were considered significant if P values were less than 0.05.

### Example 1: CB CD14⁺ Monocytes are Essential for the Production of DUOC-01 Cells

To test the hypothesis that the DUOC-01 product is derived from CD14⁺ monocytes in the CB MNC population, CD14⁺-enriched and CD14⁺-depleted cell populations from the same CB MNC fraction were simultaneously produced with immunomagnetic beads. Flow cytometric analysis showed that positively selected populations contained greater than 90% CD14⁺ cells and depleted populations contained less than 2% CD14⁺ cells. The CD14⁺ and CD14⁺-depleted cell populations were then cultured using the standard protocols for manufacture of DUOC-01 and compared the evolution of different cell types with cultures of CB MNCs. Six experiments were performed with fresh and 3 with cryopreserved, thawed CB units with essentially identical results. CD14⁺-depleted cell populations did not give rise to the cell characteristic of DUOC-01. Instead, small blood cells persisted, and very few adherent cells were present. In contrast, CB CD14⁺ monocyte populations gave rise to cultures that were morphologically indistinguishable from, and expressed surface markers characteristic of, DUOC-01.

To test the possibility that CD34⁺ hematopoietic progenitor cells could give rise to DUOC-01 cells during manufacturing, similar experiments were carried out using immunomagnetically selected CD34⁺ CB cells and CD34⁺-depleted populations. In 6 experiments using fresh and 3 using cryopreserved CB, CD34⁺ cells survived poorly, and no cells resembling DUOC-01 arose in culture (data not shown). In contrast, CD34⁺-depleted cell populations gave rise to normal numbers of DUOC-01 cells.

Because the CD14⁺ cell population is essential for the generation of the DUOC-01 cell product, the ability of freshly isolated CB CD14⁺ monocytes and 21 day-cultured DUOC-01 cell products to influence remyelination of the CC region in CPZ-fed mice was compared. To increase survival of human cells used for treatment in this xenogeneic model, the immune-incompetent NSG mice were used for CPZ-mediated demyelination and remyelination studies.

### Example 2: CC Region of NSG Mice was Severely Demyelinated and Disorganized Following CPZ Feeding

Because different mouse strains may respond to CPZ feeding in significantly different ways and because NSG mice have not previously been used in this model, the process of demyelination and remyelination of the CC in NSG animals in the absence of cell therapy was evaluated. Very similar results were obtained in each of 4 experiments. Similar to C57BL/6 mice fed 0.2% CPZ for 5 weeks, NSG mice weighed 12%-16% less than mice on normal diets and gained weight similarly to the control strain when they were returned to the normal laboratory chow after 5 weeks of CPZ feeding. Neither CPZ feeding nor injection of any of the 3 cell populations induced any obvious changes in overall behavior or general health of the animals.

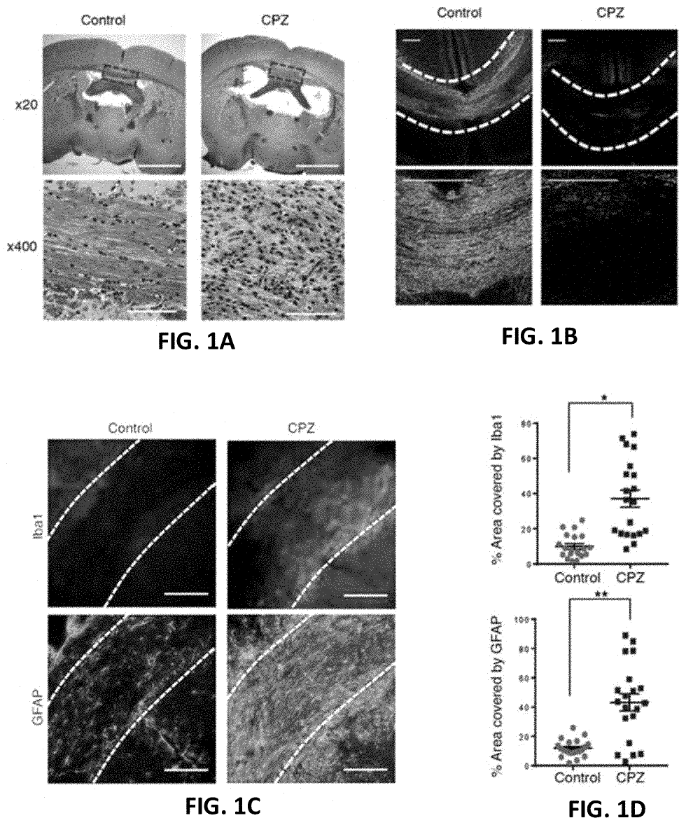

After 5 weeks of CPZ feeding, the extent of myelination in the CC was examined by staining coronal brain sections with Luxol fast blue-periodic acid Schiff (LFB-PAS). The CC region of NSG mice was severely demyelinated, with gliosis following CPZ feeding compared with control mice on standard chow (FIG. 1A). FIG. 1A also shows that peripheral regions of the CC were less affected. Thus, NSG mice exposed to CPZ demyelinate in the CC, similarly to what has been reported for C57BL/6 mice.

Immunohistochemical analysis confirmed morphological and cellular effects of CPZ feeding. The midline CC region of the brains of mice fed CPZ for 5 weeks showed very little or no staining for myelin basic protein (MBP) compared with uniform MBP staining in control animals (FIG. 1B). Astrocytes positive for glial fibrillary acidic protein (GFAP) and microglia positive for Iba1 were much more profuse in the CC region of CPZ-fed animals than those of controls (FIG. 1C), indicative of severe gliosis. Expression of both GFAP and Iba1 per unit surface area in the CC region was significantly greater in the CPZ-treated mice than in the control mice (FIG. 1D).

Electron microscopic analysis of the CC also confirmed that CPZ caused severe demyelination and showed additional disruptions of axonal structure in the region (data not shown). Spontaneous remyelination kinetics of NSG mouse brains after CPZ withdrawal was evaluated by LFB-PAS staining. 1 week after CPZ withdrawal, most of the midline CC area of NSG mouse brains remained severely demyelinated (data not shown). However, 2 weeks after CPZ withdrawal, the midline CC area of the brain was significantly remyelinated (data not shown). Thus, the effects of CPZ feeding on the CC region of NSG mice are generally similar to the effects in the more commonly used C57BL/6 mouse strain. This model was used to explore the effects of DUOC-01 treatment on the kinetics of remyelination once CPZ feeding was terminated.

### Example 3: DUOC-01 Cells Disseminated from the Injection Site and Persisted in the Brain for Up to 1 Week after Intracranial Injection

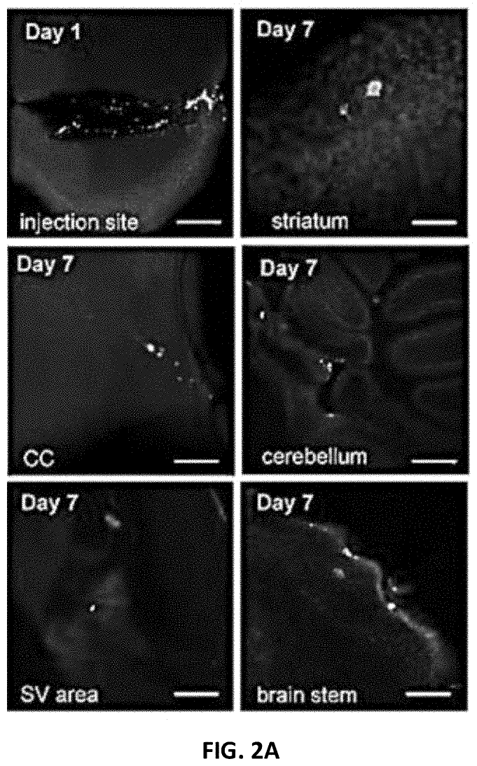

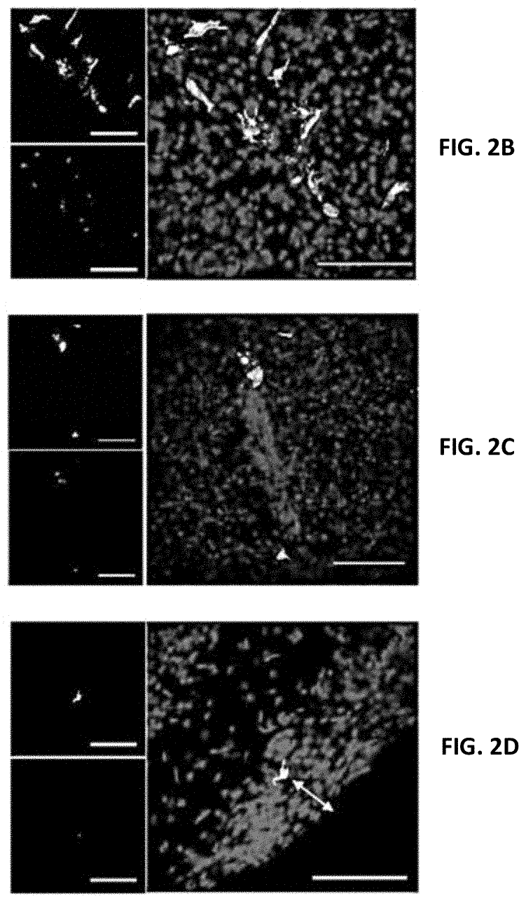

To trace human cells in the brain following stereotactic injection in the midline CC area, NSG mice that had been fed CPZ for 5 weeks were injected intracranially with 1.0×10⁵ CF SE-labeled DUOC-01 cells. CFSE stains the cells fluorescent green, and the dye is stable for weeks in vivo. CF SE-labeled cells were found at the injection site as well as in the striatum, CC, cerebellum, brain stem, and subventricular area up to 7 days after injection (FIG. 2A). To further confirm that the CFSE-positive cells observed in brain sections were injected DUOC-01 cells, immunostaining with an antibody that specifically detects human nuclei (anti-HuN) was performed. Mouse cells in the brain sections were not positive for this anti-HuN antibody. In contrast, the CFSE-positive cells costained with anti-HuN (FIG. 2B), confirming that the CF SE-stained cells were not mouse brain cells that might have taken up CFSE released by DUOC-01 or stained human cell debris. CSFE- and HuN-costained DUOC-01 cells were detected deep in the brain parenchyma and as far from the CC injection site as the frontal cortex, and persisted for up to 1 week until assessment of myelination (FIG. 2C). CFSE-stained CD14⁺ cells were also found in various parts of the brain even after 7 days after intracranial injections (data not shown). Thus, DUOC-01 cells disseminated bilaterally from the injection site and persisted in the brain during the 1-week period between cell injection and harvesting brains for assessment of myelination status.

### Example 4: DUOC-01 Treatment Accelerates Remyelination after CPZ Feeding in the CC Region of NSG Mice

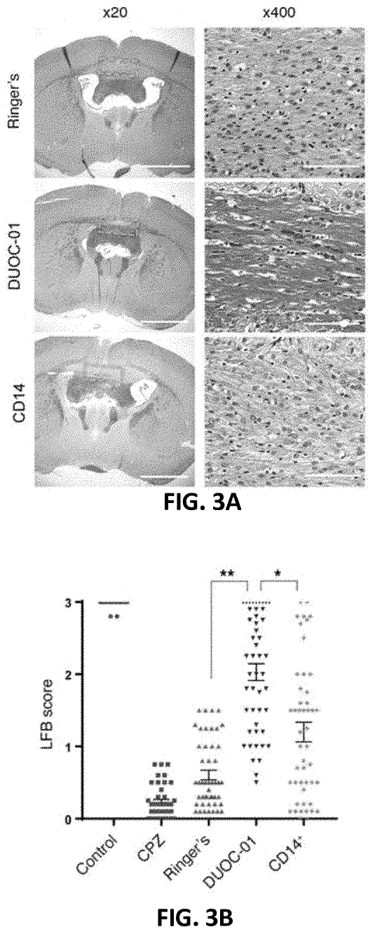

As noted above, LFB-PAS staining showed that NSG mice spontaneously remyelinated the CC region during 2 weeks following termination of CPZ feeding. In all 4 experiments, the CC of CPZ-fed mice treated with Ringer's remained severely demyelinated 1 week after diet change and injection (FIG. 3A). In contrast, LFB-PAS staining showed extensive myelin fiber formation in the CC 1 week after treatment with DUOC-01 (FIG. 3A). Myelination scores of the CC of DUOC-01-treated mice were significantly higher than those of the Ringer's-injected group (FIG. 3B). CD14⁺ cell-treated mice also showed an increased amount of remyelination compared with the Ringer's control group, but significantly less than the DUOC-01-treated group (FIGS. 3A-3B). The effects of DUOC-01 treatment in remyelination was examined in more detail.

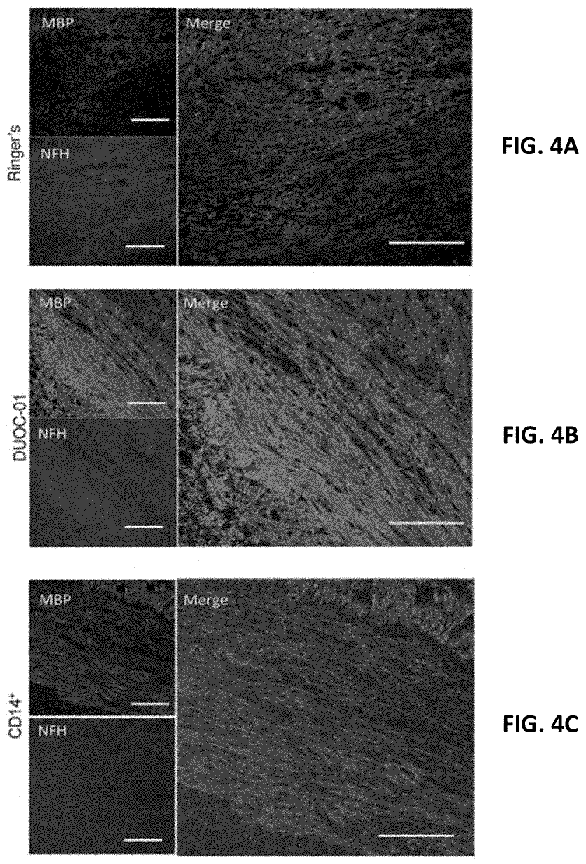

Immunohistochemical analysis with an anti-MBP antibody confirmed that DUOC-01-treated mice remyelinated more extensively than Ringer's control animals during the week after diet change and treatment (FIG. 4A). Analysis of higher magnification confocal images revealed a higher density and level of organization of MBP-containing fibers in the CC of DUOC-01-treated mice (FIG. 4B), and MBP appeared to colocalize with neurofilament-H (NFH) (FIG. 4B), indicative of myelin wrapping along the axonal fibers.

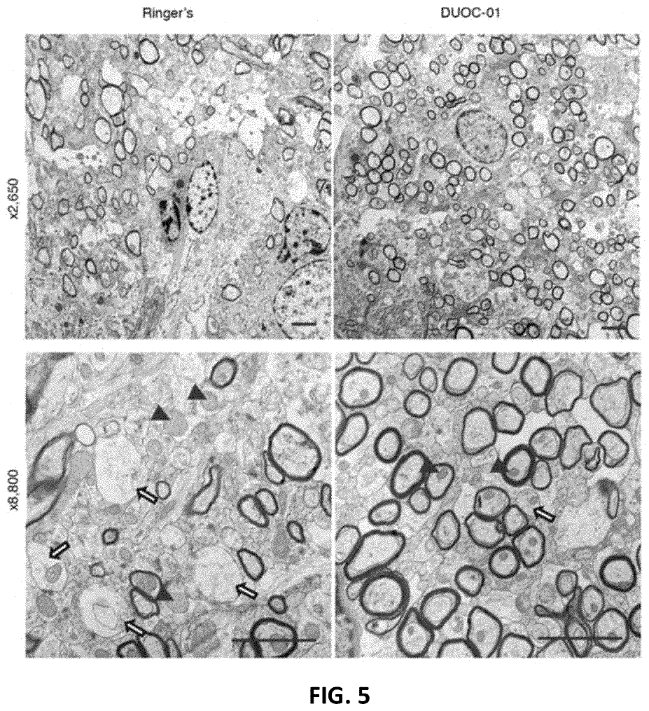

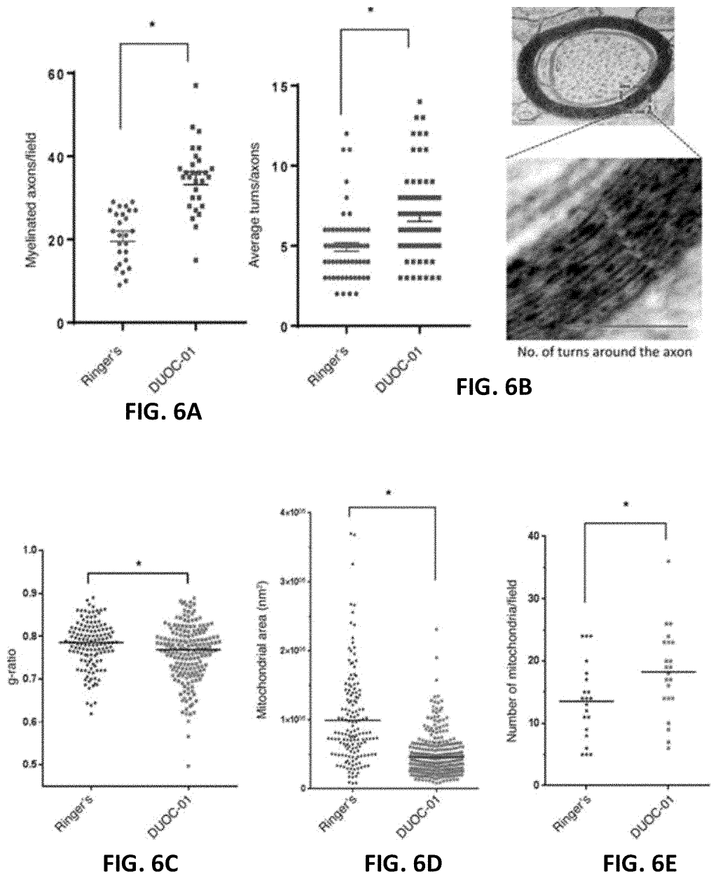

Electron microscopic analysis revealed that the newly synthesized myelin detected by immunohistochemistry in the CC of DUOC-01-treated mice was organized into myelin sheaths on axons (FIG. 5). Morphometric analysis revealed that the CC of DUOC-01-treated mice had significantly more myelinated axons than the CC of animals treated with Ringer's (FIG. 6A). To further assess the organization of the myelin sheath, the number of turns of myelin sheath wrapped around the axons was counted. The DUOC-01-treated group had approximately 2 additional turns of myelin sheath per axon compared with the control group (FIG. 6B). The g-ratio (ratio of the inner axonal diameter to the total outer [including myelin wrap] diameter) value was lower in DUOC-01-treated compared with the Ringer's-treated mice, indicating increased myelin thickness in DUOC-01-treated mice (FIG. 6C). Axonal diameters displayed a similar distribution of higher and lower g-ratios across various axon diameters both in Ringer's- and DUOC-01-treated groups. Axonal density, measured as the number of axons present per microscopic field (×8,800 magnification) of electron micrographs, was not significantly different (P<0.075) in the DUOC-01-treated and the Ringer's-treated samples (data not shown). Taken together, these data show that relative to Ringer's treatment, administration of DUOC-01 cells increased the number of remyelinated axons and augmented the myelin thickness and organization in the CC in the 7 days following treatment.

Morphometric analysis also showed that treatment with DUOC-01 accelerated the reversal of megamitochondria formation (FIG. 5). One week after DUOC-01 cell treatment, the average size of mitochondria in the brain cells of Ringer's-treated mice was significantly larger than in cells of the DUOC-01-treated group (FIG. 6D). Electron micrographs (data not shown) show that mitochondria of DUOC-01-treated brains were similar in size to those in unmyelinated control brains. In the Ringer's-treated group, enlarged mitochondria were present in both axons as well as in other cells, possibly in oligodendrocytes. Brains from DUOC-01-treated mice had a greater number of mitochondria per electron microscopic field than brains from Ringer's-treated animals (FIG. 6E). This reduction in mitochondrial size coupled with the observed increase in megamitochondria formation and larger numbers of mitochondria suggests that DUOC-01 cells helped restore mitochondrial activity during remyelination.

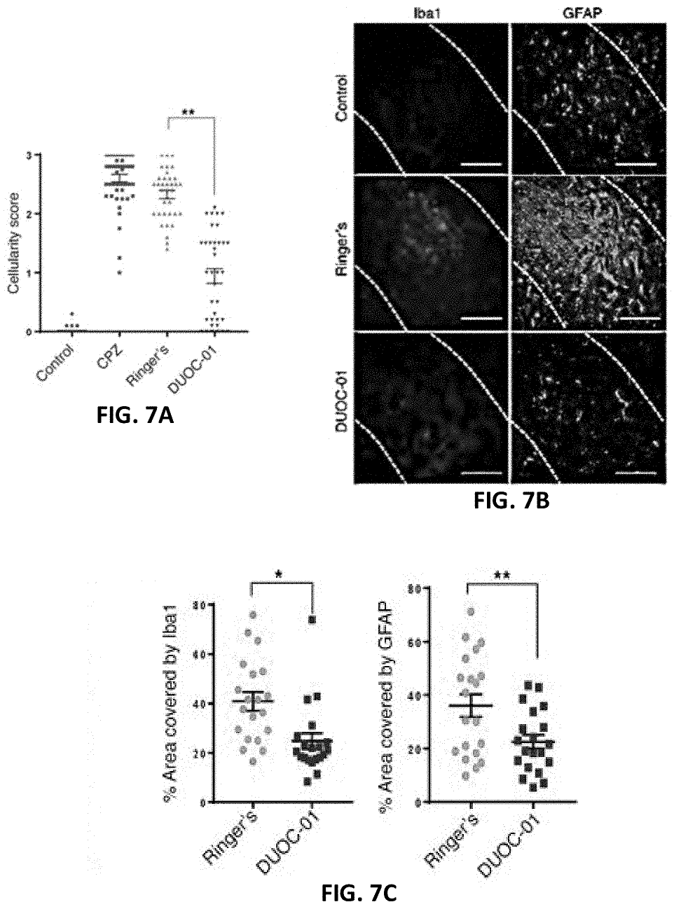

Cellularity scoring of LFB-PAS stains of CC sections showed that DUOC-01 treatment also significantly reduced cellular accumulation and gliosis in the CC region 1 week following diet change (FIG. 7A). Reduced glial accumulation was also evident in brain sections stained with antibodies against GFAP to detect astrocytes and Iba1 to detect microglia (FIG. 7B). Quantitative analysis of areas covered by Iba1- and GFAP-positive cells was performed, indicative of their numbers, along the CC. Both the numbers of Iba1-positive (microglia) and GFAP-positive (astrocytes) cells were significantly lower in the CC area of the DUOC-01-treated animal brains (FIG. 7C). The cellularity score also was decreased in the CD14⁺ cell-injected group compared to the Ringer's-injected control, but not as markedly as the DUOC-01-treated group.

### Example 5: DUOC-01 Cell Treatment Promotes Oligodendrocyte Progenitor Proliferation

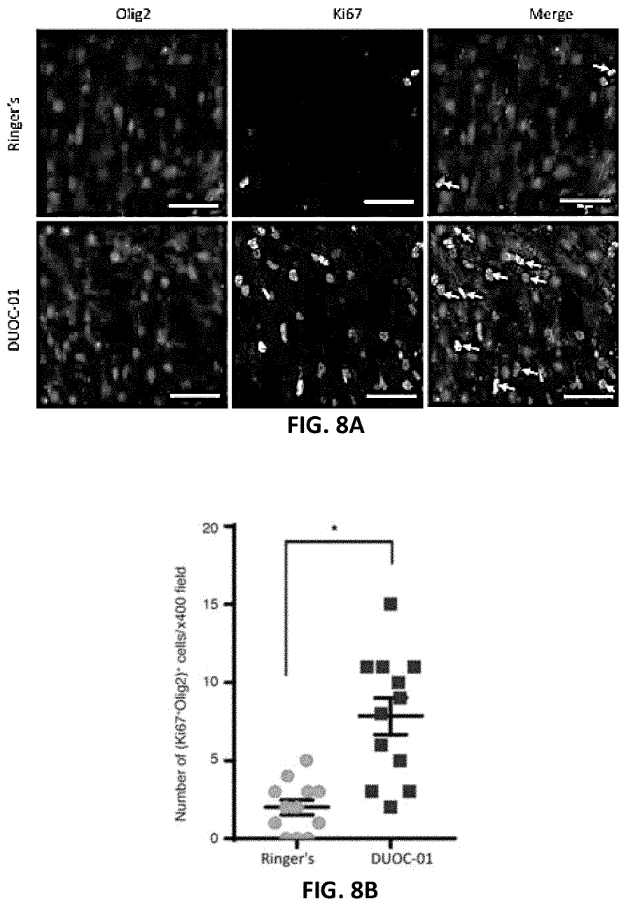

Next it was determined whether DUOC-01 treatment increased the number of proliferating oligodendrocyte progenitor cells in the CC area following cessation of CPZ feeding (FIG. 8). In adult brains, the oligodendrocyte lineage transcription factor 2 (Olig2) is present in the nuclei of oligodendrocyte progenitors and mature oligodendrocytes. Ki67 is only present in proliferating cells. Thus, the combination of anti-Olig2 and anti-Ki67 antibodies was used to detect newly generated cells in the oligodendrocyte lineage. The number of proliferating Olig2⁺ Ki67⁺ oligodendrocytes (FIG. 8B) present in the CC region was significantly higher in brain sections from DUOC-01-treated animals than in controls that did not receive cell therapy. There was no significant increase in the number of proliferating Olig2⁺ Ki67⁺ oligodendrocytes in CB CD14⁺-treated brains as compared with the Ringer's-injected group (data not shown). Thus, DUOC-01 treatment promotes oligodendrogenesis, which in turn could facilitate remyelination.

### Example 6: Identification of Gene Products Expressed by DUOC-01 that May Promote Remyelination

Whole-genome expression microarrays was used to identify candidate DUOC-01 genes that may participate in acceleration of remyelination of the CC following CPZ feeding. As DUOC-01 cells promote remyelination much more robustly than CB CD14⁺ monocytes, the initial strategy was to identify differentially expressed transcripts that were more abundant in DUOC-01 than in CB CD14⁺ monocytes. Whole-genome microarray analysis was performed on 4 highly purified, flow cytometry-sorted, CB CD14⁺ monocytes and 3 DUOC-01 cell products. Complete expression data have been deposited in the NCBI's Gene Expression Omnibus (GEO GSE76803).

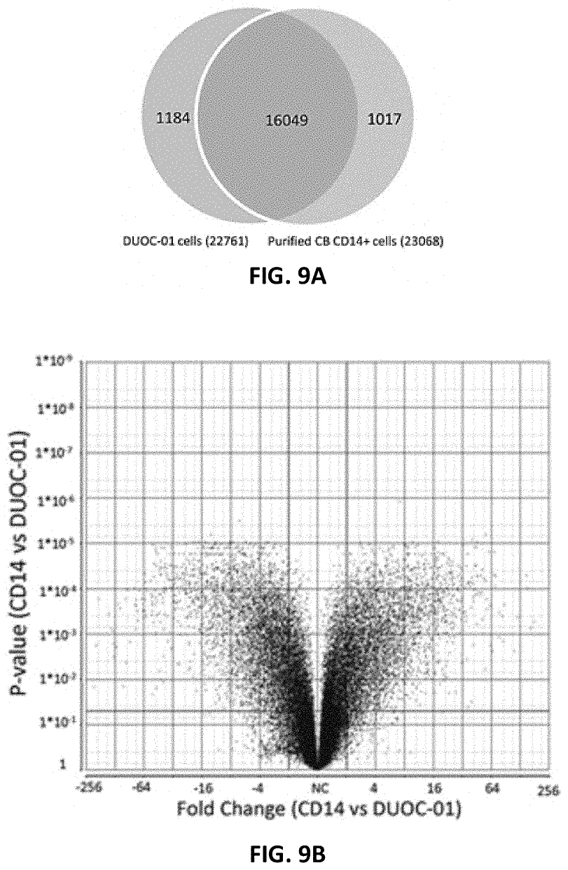

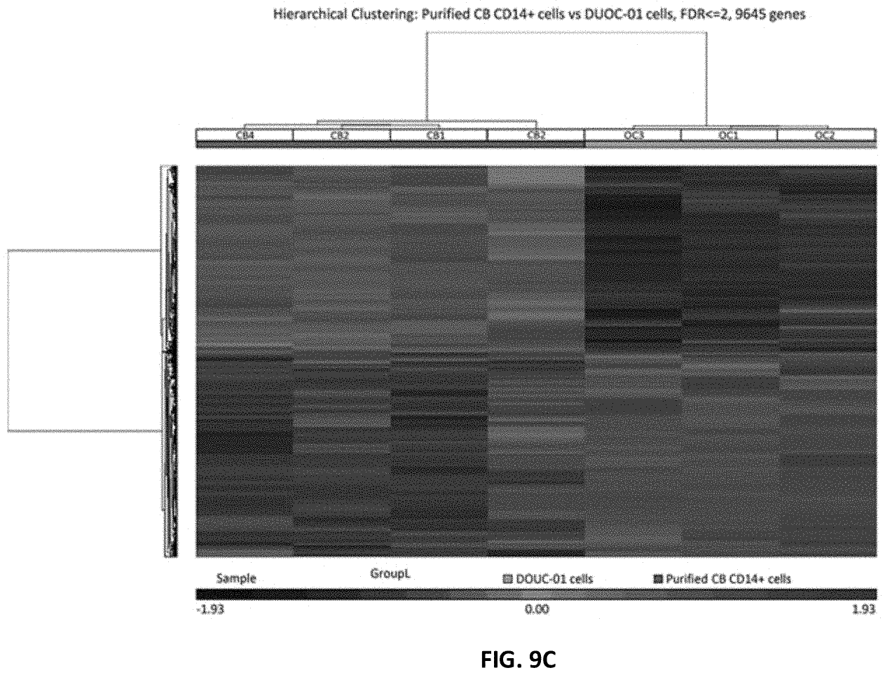

Stringent MASS analysis was used to identify expressed genes. In order for a transcript corresponding to a probe to be scored as “present,” all 4 CB CD14⁺ cell samples or all 3 DUOC-01 samples were required on the chip showed expression with a specific probe set. A Venn diagram displaying the findings from microarray analysis showing the number of genes only expressed in purified fresh CD14⁺ or DUOC-01 cells as well as genes expressed by both cell types is shown in FIG. 9A. For less stringent analysis, transcripts that were not detected in at least 1 sample, but detected in others were scored as “mixed,” and transcripts absent in all samples were scored as “absent.” The 2 cell populations differed considerably in gene expression. Thus, 1,184 probe sets detected transcripts in all DUOC-01 samples that were absent in all CB CD14⁺ monocyte samples and, conversely, 1,017 transcripts were present in all CB CD14⁺ monocytes and absent in all DUOC-01 samples. In addition, 3,189 probe sets detected transcripts in 1 or 2 of the 3 DUOC-01 lots but none of the 4 CB CD14⁺ preparations. Conversely, 3,496 probes detected transcripts in 1, 2, or 3 of the 4 CB CD14⁺ preparations but none of the DUOC-01 lots. Additional differences in expression were observed when requirements were less stringent.

Quantitative changes in the level of expression of transcripts expressed by both cell populations when CB CD14⁺ monocytes differentiated into DUOC-01 were also analyzed. Lists of expressed genes were generated using the Partek software suite, with stringency set such that a probe was scored as expressed if 3 or 4 of the CB CD14⁺ cell samples showed expression or 2 or 3 of the DUOC-01 samples showed expression. ANOVA was performed on robust multiarray average-normalized (RMA-normalized) expression levels, and all genes that showed greater than 2-fold differences in expression between the 2 groups were identified and selected for further analysis. Altogether, 8,566 probes detected transcripts that were significantly (P<0.05) expressed at least 2-fold differently between the 2 populations. Of these, 3,585 probes detected transcripts expressed at higher levels in CB CD14⁺ monocytes, and 4,979 probes detected transcripts more highly expressed in DUOC-01. The volcano plot in FIG. 9B graphically represents the marked difference in the gene expression pattern between the 2 cell types. Tabular representation of Pearson's correlation coefficients among the samples showed a lower correlation (0.87-0.90) among the CB CD14⁺ and DUOC-01 groups, although the correlation coefficient was much higher (0.97-0.99) between the samples within the same group. Differentially expressed transcripts are listed in GEO GSE76803. The heat map presented in FIG. 9C also demonstrates that DUOC-01 and CB CD14⁺ cells fall into discrete populations defined by a large number of differentially expressed transcripts.

To annotate the function of genes that were differentially expressed, uncharacterized transcripts, pseudogenes, and non-protein-coding transcripts were eliminated from further analysis. The resulting gene lists were examined using the tools aggregated at the DAVID website. The functional cluster analysis for the genes that are more highly expressed in DUOC-01 cells than CB CD14⁺ monocytes was performed. The list was enriched for genes involved in all aspects of cell division and mitosis. Pathway analysis also showed an abundance of genes involved in cell division and in lysosomal activity and trafficking of intracellular vesicles. Of interest, genes encoding factors previously shown to be secreted and/or increased (e.g., IL-10, TGF-β, and galactocerebrosidase [GALC]) during manufacturing of DUOC-01, were identified in the DUOC-01 upregulated gene list. In addition, transcripts for several other lysosomal enzymes that are secreted by DUOC-01 are more abundant in the cell product than in CB CD14⁺ cells, and pathway analysis shows a very high probability of enrichment for genes encoding proteins in the lysosomal lumen in DUOC-01. A variety of transcripts that can influence myelination are highly overexpressed in DUOC-01 cells relative to CD14⁺ cells. In contrast, the list of genes more highly expressed in CB CD14⁺ cells was enriched in transcription factors and signaling molecules, particularly in repressors of transcription. Genes active in hematopoiesis and myeloid cell differentiation are also more common. Genes active in mitosis and cell cycle entry are much less common than in the list derived from DUOC-01.

Some of the transcripts overexpressed in DUOC-01 that are known to be important in promoting remyelination were selected and their level of expression in CB CD14⁺ and DUOC-01 cells was confirmed by quantitative PCR methods. Expression of these candidate molecules is presented in Table 1. Platelet-derived growth factor subunit A (PDGFA), KIT-ligand (KITLG, also known as stem cell factor [SCF]), insulin-like growth factor-1 (IGF1), triggering receptor expressed on myeloid cells 2 (TREM2), matrix metalloproteinase-9 (MMP9), and MMP12 were highly upregulated in DUOC-01 cells compared with CB CD14⁺ monocytes; bioplex analysis also demonstrated that DUOC-1 cells secrete MMP9, MMP12, and other matrix proteases into culture supernatants (unpublished observation). IL10 transcript levels were also enriched in DUOC-01 cells. Western blot analysis confirmed enrichment of TREM2, SCF, MMP9, and MMP12 proteins in DUOC-01 relative to CB CD14⁺ monocyte homogenates. Higher expression of TREM2 on the DUOC-01 cell surface was also verified by flow cytometry. However, the relative abundance of IGF1 and PDGF-AA protein detected by Western blotting did not replicate transcript abundance. IGF1 and PDGF-AA proteins were detected in CD14⁺ CB monocytes but not in DUOC-01 homogenates. Without being bound to a theory, it is hypothesized that failure to detect IGF1 and PDGF-AA in DUOC-01 homogenates might result from their rapid secretion from the cells. To test this idea, DUOC-01 cells were allowed to adhere to glass slides and then incubated for 5 hours with brefeldin A (BFA). BFA treatment rapidly inhibits transport of secretory proteins from the ER to the Golgi, resulting in accumulation of proteins within the ER. Western blot analysis of DUOC-01 cells after BFA treatment showed higher intracellular concentrations of both IGF1 and PDGF-AA, indicating that these proteins are rapidly secreted by DUOC-01 cells. Immunocytochemical analysis of BFA-treated DUOC-01 cells using IGF1 and PDGF-AA antibodies also showed higher levels of staining after BFA treatment compared to cells without any BFA pretreatment. These data suggest that DUOC-01 cells express and secrete factors known to promote remyelination by several mechanisms and enhance oligodendrocyte precursor proliferation and differentiation.

TABLE 1
Quantitative PCR determination of abundance of transcripts
of factors that promote oligodendrogenesis and myelination
in DUOC-01 and CB CD14⁺ monocytes

| Gene name | Fold change (Mean ± SEM, n > 3) | P value |
|---|---|---|
| PDGFA | 32.3 ± 8.3 | ≤0.01 |
| KITLG/SCF | 26.7 ± 4.8 | ≤0.033 |
| IGF1 | 799 ± 294 | ≤0.05 |
| MMP9 | 632 ± 109 | ≤0.002 |
| MMP12 | 2057 ± 460 | ≤0.006 |
| TREM2 | 1634 ± 368 | ≤0.011 |

Results

The studies presented here confirm that the DUOC-01 cell product of the disclosure strongly promotes remyelination in an animal model that does not depend on enzyme replacement: CPZ-induced demyelination of the CC. Injecting DUOC-01 cell product into the CC area 1 day after CPZ-fed NSG mice were returned to normal diets dramatically accelerated reversal of all the pathological manifestations of CPZ feeding during the following week. The course of demyelination, astrogliosis, microgliosis, cellular accumulation, and the reversal of these processes following cessation of CPZ feeding in NSG mice closely resembled what has been reported in C57BL/6 mice. Using CFSE-labeled DUOC-01 cell product, it was found that DUOC-01 cells reached brain regions remote from the injection site and that cells could be detected in the brain throughout the 1-week experiment. Storms et al. (Cytotherapy 2014; 16(4):563) have reported that a low percentage of intrathecally injected DUOC-01 cells persist in the brain of neonatal NSG mice for up to 56 days. Immunohistochemical and LFB-PAS staining demonstrated that DUOC-01 treatment accelerated remyelination of the CC, and electron microscopy revealed that administration of DUOC-01 cells increased the proportion of remyelinated axons and the thickness and organization of the myelin sheath following treatment. DUOC-01 treatment also significantly resolved gliosis in the CC induced by CPZ feeding. Finally, electron microscopic analysis also showed that DUOC-01 treatment reduced the number of megamitochondria in the CC region, suggesting that DUOC-01 treatment reverses metabolic stress, abnormality of mitochondrial fission, or oxidative stress induced by CPZ treatment. Thus, DUOC-01 cells can deploy within the cerebral cortex following intracerebral injection and accelerate remyelination after cessation of CPZ feeding. During manufacture of DUOC-01 from CD14⁺ CB monocytes, expression of many factors that can influence remyelination by a variety of mechanisms is upregulated. These studies provide proof of concept for the clinical use of DUOC-01 in the treatment of demyelinating diseases.

Freshly isolated CB CD14⁺ monocytes also accelerated remyelination and reduced cellular infiltration in the CC following discontinuation of CPZ feeding, but did so significantly less markedly than DUOC-01 cells. In addition, DUOC-01 treatment increased proliferation of oligodendrocyte lineage cells, but treatment with CD14⁺ CB monocytes did not.

CB CD14⁺ monocytes are essential for the manufacture of DUOC-01 cells implying that changes in gene expression occurring during manufacturing enhance the ability of DUOC-01 cells to promote remyelination. Whole-genome expression analysis demonstrated that CB CD14⁺ monocytes and DUOC-01 differed in their expression of thousands of transcripts, and subsequent studies based on real-time PCR, Western blotting, and flow cytometry confirmed that CB CD14⁺ monocytes and DUOC-01 differ significantly in expression of some secretory proteins and other molecules that can promote remyelination following CPZ feeding by multiple mechanisms. The differences in gene expression between CD14⁺ CB monocytes and DUOC-01 cell products were used as an initial screen to identify molecules that may play important mechanistic roles in accelerating remyelination with several caveats. First, transcript levels need not reflect levels of protein production. Indeed, while both proteins and transcripts for SCF, TREM2, MMP9, and MMP12 were highly expressed in DUOC-01 but not expressed in CD14⁺ CB monocytes, IGF1 and PDGF-AA proteins were expressed in both cell types at similar levels in spite of differences in transcript levels. Second, some molecules that are expressed at similar levels by both cell types could play important roles in remyelination by both cell types; such molecules would not be detected if only differentially expressed transcripts were considered as candidates. The differentially expressed transcripts that was detected provide markers for more in-depth analysis of changes in injured tissue. Thus, the gene expression data provide a starting point to elucidate the mechanisms by which DUOC-01 promotes remyelination.

Several of the proteins that are expressed by DUOC-01 cells are known to regulate the number or activity of oligodendrocyte progenitor cells (OPCs). PDGFs regulate the OPC numbers in the adult CNS and their activity following CNS demyelination, and PDGFA transcript expression was upregulated 32-fold in the DUOC-01 compared with CB CD14⁺ monocytes. SCF has been implicated in the maintenance, migration, and survival of the OPC population, and its transcript was expressed at a 26-fold higher level in DUOC-01 cells than in CB CD14⁺ cells. Similarly, expression of IGF1 transcripts was almost 800-fold higher in DUOC-01 compared to the CD14⁺ cells. IGF1 has been shown to induce myelination in vitro and in vivo and also protects mature oligodendrocytes from a pathological insult. Furthermore, IGF1 promotes the long-term survival of mature oligodendrocytes in culture and inhibits mature oligodendrocyte apoptosis in vitro. Brefeldin A-mediated inhibition of secretory proteins demonstrated that PDGF-AA and IGF1 are both rapidly secreted from DUOC-01 cells. These factors, then, could directly drive the large increase in proliferating oligodendrocyte lineage cells that was observed in the CC of DUOC-01-treated animals compared with controls receiving no cell therapy.

TREM2 is another molecule expressed by DUOC-01 cells that plays an important role in remyelination. CD14⁺ monocytes do not express TREM2 transcripts or protein. This surface receptor senses lipid debris and regulates signaling by glial cells that modulate myelination. It also functions in clearance of cellular and myelin debris, an important early step for recovery and remyelination following CNS injury. DUOC-01 cells are highly phagocytic and could play a significant role in myelin clearance and intercellular signaling through the TREM2 receptor. DAVID analysis of differentially expressed genes showed that the lysosomal/intracellular vesicular pathway and Fcγ-mediated phagocytosis were among the most highly upregulated group of genes in DUOC-01 compared with CD14⁺ CB monocytes.

DUOC-01 cell product of the disclosure expresses many other proteins that could participate more indirectly in promoting remyelination and in resolving cellular accumulation in the CC. Cytokine-activated microglia can stimulate the differentiation of oligodendrocytes from neural progenitor cells. While oligodendrocytes affect the remyelination of nerve fibers, other cell types are important for this repair process. Astrocytes provide trophic factors for oligodendrocytes and also for microglia. Microglia also provide trophic factors and remove myelin debris that inhibit remyelination by oligodendrocytes. The microarray data indicate that several chemokines and other regulators of neuroinflammation are upregulated in DUOC-01 cells. It was previously reported that DUOC-01 cells secrete IL-10 and TNF-α in culture (Kurtzberg J, et al. Cytotherapy. 2015; 17(6):803-815). Yang et al. have demonstrated that neuronal stem cells producing IL-10 not only effectively suppress CNS inflammation but also promote remyelination and neuron/oligodendrocyte repopulation in a mouse model of experimental autoimmune encephalomyelitis (J Clin Invest. 2009; 119(12):3678-3691). Furthermore, IL-10 promotes survival of neurons and oligodendrocytes by protecting them from inflammation-induced damage. It has been shown that TNF-α plays an important reparative role in the demyelinating brain. Lack of TNF-α led to a reduction in the pool of proliferating OPCs and subsequent significant delay in remyelination in CPZ-mediated demyelinated brain (Arnett H A, et al. Nat Neurosci. 2001; 4(11):1116-1122). The microarray data indicate that several other factors including chemokines and other regulators of neuroinflammation are upregulated in DUOC-01 cell product of the disclosure.

DUOC-01 cells also overexpress proteases that can regulate remyelination through modification of the extracellular matrix. Upregulation of MMP9 and MMP12 was confirmed by PCR and Western blotting. CD14⁺ CB monocytes did not detectably express either protease. MMP9 activity is required to clear NG2 chondroitin sulfate proteoglycan deposition and overcome the negative impact of NG2 on oligodendrocyte maturation and remyelination (Larsen P H, et al. J Neurosci. 2003; 23(35):11127-11135). High expression of MMP12, which is required for proteolysis and matrix invasion by macrophages in mice, might facilitate the migration of DUOC-01 from the injection site in the CC to other regions of the brain that was observed using CFSE-labeled cells. MMPs also play a role in angiogenesis, in the release of growth factors sequestered by the extracellular matrix, and in processing of cell-cell recognition molecules that allow repair.

These studies constitute proof of concept that the monocyte-derived DUOC-01 cell product of the disclosure provides benefit to patients with demyelinating conditions. In certain embodiments, the demyelinating conditions may be selected from leukodystrophies, multiple sclerosis, or spinal cord injury.

### Example 7: General Strategy and Pilot Analysis of Kinetics of Gene Regulation During Manufacturing

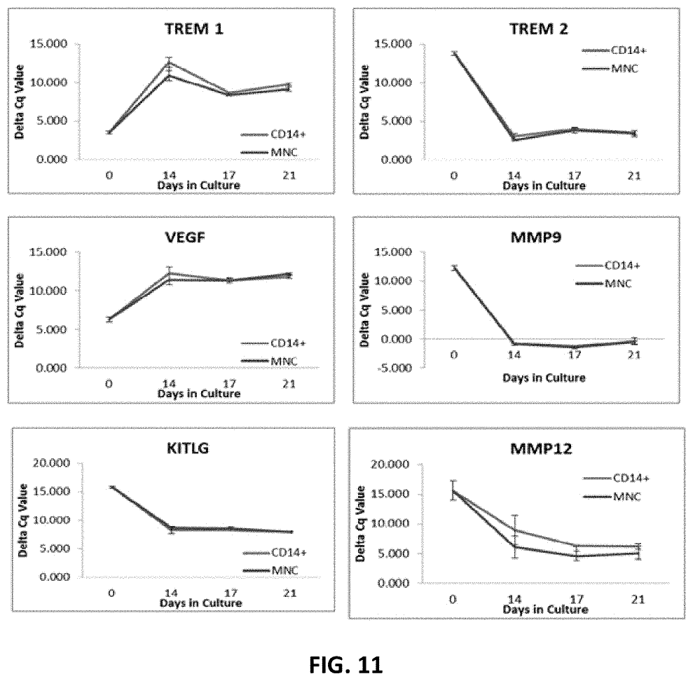

The primary goal of these studies was to gather more information related to possible mechanisms of action of DUOC-01 cells in brain remyelination. In addition, the probes that reflect expression of mechanistically important gene products was developed to monitor manufacturing of this cell product, assess activity and product potency and determine product comparability following manufacturing changes. To control for inherent biological variability among CB units, cell products made from portions of the same CB unit under different conditions were compared rather than comparing products made from different units during this manufacturing-oriented work. This placed limits on the number of variables that could be explored with a single unit. Consequently, before proceeding with full scale studies, a small pilot experiment was first performed to determine how rapidly characteristic changes in gene expression could be detected during the DUOC-01 manufacturing process and whether these changes followed the same kinetics in cultures initiated with CD14+ monocytes instead of MNC. On the basis of the previous morphological studies of DUOC-01 in culture and the yield of cells derived at various times during manufacturing, the analysis began on day 14 of manufacturing just before the first medium change, on day 17 just before the medium was changed again, and when the cell product was harvested on day 21 (FIG. 10). qPCR was used to analyze expression of six genes that were differentially expressed in the previous microarray studies demonstrating that TREM1 and VEGF were down-regulated in cultured DUOC-01 cell products relative to freshly isolated CD14+ monocytes, and TREM2, KITLG, MMP9 and MMP12 were up-regulated. The results (FIG. 11) confirmed these changes in the abundance of transcripts for the six genes. In addition, FIG. 11 shows that medium changes on days 14 and 17 during manufacturing had no detectable effect on the abundance of these six transcripts. Regulation of all six gene products appeared to be complete by day 14 of manufacturing whether cultures were started with MNCs or with CD14+ monocytes. Only the activities of cell products cultured for 14 and 21 days were compared in subsequent full-scale studies. Thus, the experiments in which gene expression by four cell products—days 14 and 21 cultures derived from either CB MNC or from CD14+ monocytes—from the same CB unit were compared.

### Example 8: Expression of 77 Transcripts Related to Remyelination During the Manufacturing Process

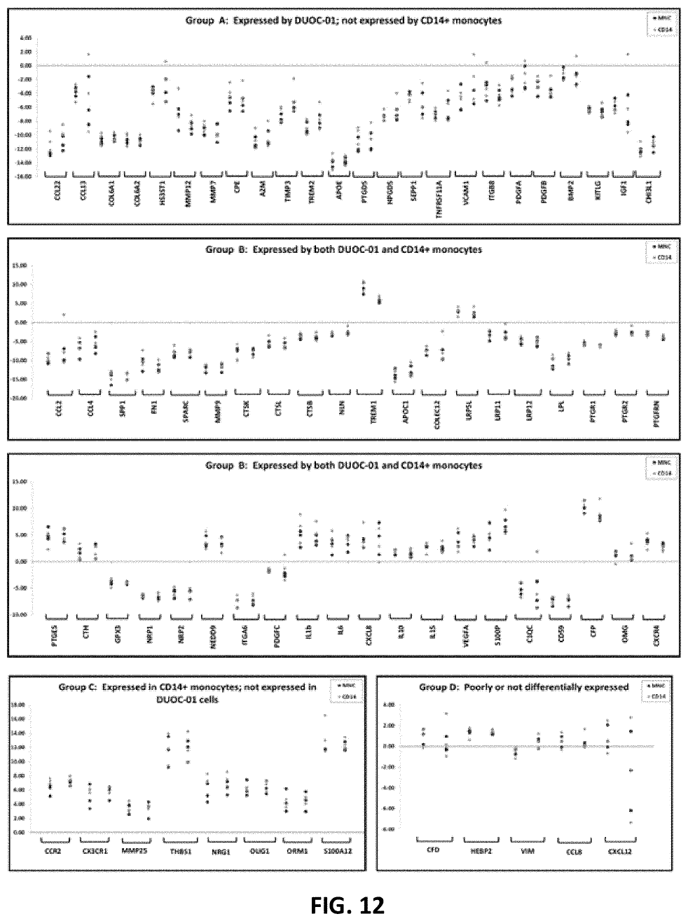

To measure expression of gene products that can contribute to remyelination by DUOC-01, a custom array for qPCR analysis was constructed. Many of the transcripts selected have multiple biological activities, but all represent potentially important activities in pathways that modulate neural or glial cell activity during brain repair or development. The array included 24 gene products that were expected to be uniquely expressed by DUOC-01 on the basis of the previous microarray study. These genes could be responsible for previously described (Saha A, et al. “A cord blood monocyte-derived cell therapy product accelerates brain remyelination.” JCI Insight 2016; 1:e86667) enhanced remyelinating activity of DUOC-01 compared with CD14+ monocytes. However, genes that are expressed by both DUOC-01 and CD14+ monocytes could also participate in remyelination, and 45 probes for such genes were included. Finally, probes for eight genes that were expected to be strongly expressed by CD14+ monocytes but not detectably expressed by DUOC-01 were added. This allowed to monitor extinction of transcripts characteristic of CB CD14+ cells as well as appearance of transcripts typical of DUOC-01 for in-process monitoring and other testing related to quality and regulatory purposes. This custom array was used t to measure expression of these 77 genes by cells in DUOC-01, that is, product manufactured from CB MNC, using the standard 21-day protocol. Gene expression was also compared by these standard MNC-derived DUOC-01 products to CB CD14+ monocyte-derived cell products from the same three cord blood units harvested after 14 and 21 days in culture. Changes in expression calculated from these data are summarized in FIG. 12. The limit which could reliably detect expression at 35 cycles was defined. Thus, any value of Ct>35 is considered not expressed, and any value of ΔCt calculated using a Ct value>35 represents a lower limit of changes in expression. In general, changes in gene expression in cells derived from all CB units changed in the same way; changes in CCL13, CXCL12, C1QC and IGF1, showed the most variability between units after 21 days in culture. As expected, 24 genes in the custom array were not detectably expressed by freshly isolated CB CD14+ monocytes genes (Group A in FIG. 12), and eight were not detectably expressed by DUOC-01 (Group C). Forty genes (Group B) were detectably, but differentially, expressed by the two cell types. Within Group B, 25 genes were more highly expressed in DUOC-01, and 15 were more highly expressed in non-cultured CD14+ monocytes. The degree of over-expression ranged from about 2-fold to more than 30,000-fold. Five genes (Group D) were either expressed at very low levels or were not differentially expressed. With the exception of the transcripts in Group D, the results in FIG. 12 generally confirm the expression differences detected by the microarray chip and allow assignment of discrete gene expression profiles to DUOC-01 cells and to the CD14+ monocytes from which DUOC-01 cells are derived during manufacturing. FIG. 12 also shows that DUOC-01 (blue) and CD14+ monocyte-derived (red) cell products harvested after 21 days in culture were highly similar with respect to gene expression profile. Cells analyzed after 14 days in culture (left data column for each gene in FIG. 12) had already changed in expression of each transcript analyzed relative to freshly isolated CD14+ cells, and little or no change in transcript abundance was detected when cells were cultured for another week (right data column for each gene). This extends the results presented for six genes in FIG. 11 to the full 77-gene custom array.

### Example 9: Accumulation of Secreted Proteins in Supernatants During Manufacturing

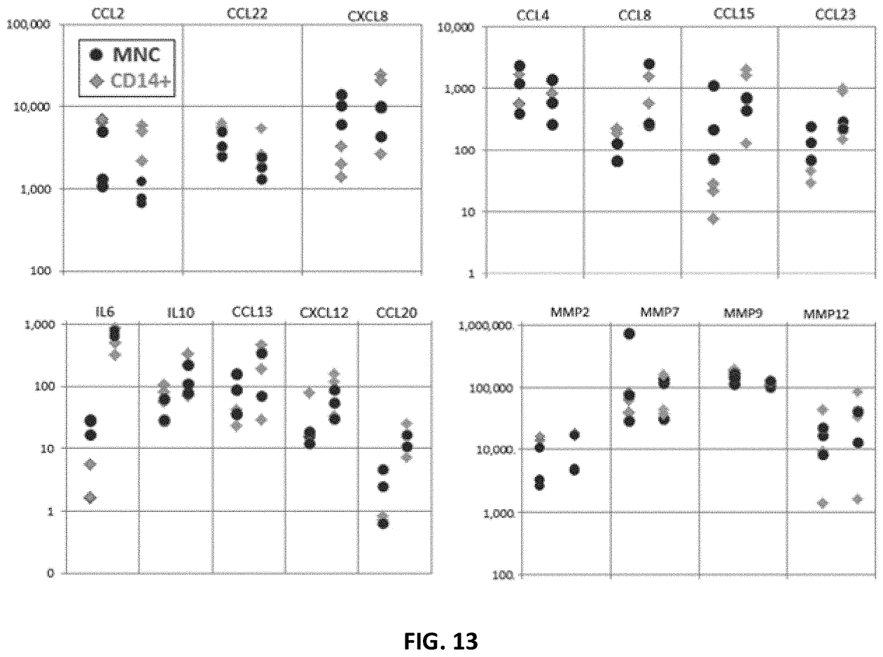

To determine how these gene expression patterns reflect production of proteins, Bioplex methods were used to measure accumulation of IL-6, IL-10 and 10 chemokines in the medium of cultures initiated with CB MNC or CB CD14+ monocytes. These culture supernatants were collected from the same cultures that were analyzed by qPCR; results are presented in FIG. 13. It was previously reported that IL-10 and IL-6 accumulated in significant amount in culture medium harvested on day 21; this result was confirmed in these three additional manufacturing batches. In addition, it was found that IL-10 could be readily detected in medium on day 14. IL-6 was present in very low concentrations at this time (FIG. 13, bottom left). qPCR data in FIG. 12 indicates that IL-6 and IL-10 are somewhat more highly expressed at the transcript level by CD14+ monocytes than by DUOC-01 cells. Nevertheless, both proteins were actively produced by DUOC-01. All 10 chemokines could be detected in the medium on days 14 and 21 of the manufacturing process. The concentration of chemokines that accumulated in the medium varied considerably (FIG. 13). CCL2, CCL4, CCL22 (all over-expressed by DUOC-01 at the transcript level; see FIG. 12) and CXCL8 (IL-8; more transcript expressed in CD14+ monocytes, FIG. 12) were present in ng/mL amounts. CCL20 was detected in the 1-20 pg/mL range, and the other chemokines accumulated in intermediate concentrations. CCL8 and CXCL12 transcripts were not differentially expressed by qPCR (FIG. 12). Similar levels of each chemokine accumulated in the medium of DUOC-01 and CD14+ monocyte-derived cell products. FIG. 13 (bottom right) shows that four matrix metalloproteases accumulated at between 2.6 and 192 ng/mL amounts in the culture medium from DUOC-01 and CD14-derived cell products. MMP7, MMP9 and MMP12 were also included in the PCR array, and all three were over-expressed in DUOC-01 products (FIG. 12). The amounts of proteases in cultures started with the two cell types were not statistically different. Again, accumulation of all of the proteins could be detected in supernatants by day 14 of manufacturing.

### Example 10: Comparison of Transcriptomes of Products Manufactured from MNC and CD14+ Monocytes

Finally, microarray methods were used to extend the expression analysis to the whole transcriptomes of DUOC-01 and of cell products manufactured from CB CD14+ monocytes derived from the same CB units. Of the 54,675 transcripts probed on the whole transcriptome chip, only 24 showed a statistically significant (P<0.05) difference in expression of more than twofold. These transcripts are listed in Table 2. The differences in expression for these transcripts were all less than six fold.

TABLE 2
Microarray analysis of transcriptomes of cell product manufactured
from MNC or CD14+ monocytes from the same CB units.

| | Gene | | Fold | P |
|---|---|---|---|---|
| Probeset ID | symbol | Gene name | Change | value |
| 205237_at | FCN1 | ficolin (collagen/fibrinogen domain containing) 1 | −5.87 | 0.019 |
| 203680_at | PRKAR2B | protein kinase, cAMP-dependent, regulatory, | −4.03 | 0.028 |
| | | type II, beta | | |
| 209392_at | ENPP2 | ectonucleotide pyrophosphatase/phosphodiesterase 2 | −3.72 | 0.014 |
| 210839_s_at | ENPP2 | ectonucleotide pyrophosphatase/phosphodiesterase 2 | −3.22 | 0.039 |
| 213194_at | ROBO1 | roundabout, axon guidance receptor, homolog 1 | −2.76 | 0.047 |
| | | (Drosophila) | | |
| 209686_at | S100B | S100 calcium binding protein B | −2.58 | 0.042 |
| 220146_at | TLR7 | toll-like receptor 7 | −2.52 | 0.045 |
| 206111_at | RNASE2 | ribonuclease, RNase A family, 2 (liver, | −2.44 | 0.034 |
| | | eosinophil-derived neurotoxin) | | |
| 202478_at | TRIB2 | tribbles homolog 2 (Drosophila) | −2.40 | 0.042 |
| 215838_at | LILRA5 | leukocyte immunoglobulin-like receptor, | −2.31 | 0.034 |
| | | subfamily A (withTM domain), member 5 | | |
| 227677_at | JAK3 | Janus kinase 3 | −2.25 | 0.050 |
| 229934_at | — | — | −2.01 | 0.002 |
| 226869_at | MEGF6 | multiple EGF-like-domains 6 | 2.00 | 0.028 |
| 203304_at | BAMBI | BMP and activin membrane-bound inhibitor | 2.03 | 0.043 |
| | | homolog (Xenopus laevis) | | |
| 229125_at | KANK4 | KN motif and ankyrin repeat domains 4 | 2.07 | 0.002 |
| 204879_at | PDPN | podoplanin | 2.17 | 0.001 |
| 1559459_at | LOC613266 | Uncharacterized LOC613266 | 2.19 | 0.039 |
| 242871_at | PAQR5 | progestin and adipoQ receptor family memberV | 2.26 | 0.011 |
| 219874_at | SLC12A8 | solute carrier family 12 (potassium/chloride | 2.55 | 0.037 |
| | | transporters), member 8 | | |
| 218469_at | GREM1 | gremlin 1, DAN family BMP antagonist | 2.88 | 0.022 |
| 209230_s_at | NUPR1 | nuclear protein, transcriptional regulator, 1 | 2.98 | 0.017 |
| 203083_at | THBS2 | thrombospondin 2 | 3.48 | 0.018 |
| 235874_at | PRSS35 | protease, serine, 35 | 3.90 | 0.013 |
| 204475_at | MMP1 | matrix metallopeptidase 1 (interstitial collagenase) | 4.56 | 0.038 |

RNA prepared from each culture was subjected to microarray analysis. The table shows all transcripts differentially expressed (greater than twofold difference, P < 0.05, not corrected for multiple comparisons) by the two populations. Positive values indicate higher expression in cultures derived from CD14+ monocytes.

Results

The most important result of this study is the demonstration that multiple pathways that can promote brain myelination after injury are activated in cells in the standard DUOC-01 cell product that is currently used in clinical trials—the product initiated with CB MNC and harvested after 21 days in culture. Of particular interest, as confirmed here by qPCR methods that 24 transcripts not detectably expressed by freshly isolated, uncultured CB CD14+ cells are highly expressed by DUOC-01 products.

The genes represented by these transcripts are prime candidates for mediating the enhanced ability of DUOC-01 to accelerate remyelination when compared with CD14+ monocytes in the cuprizone model. These transcripts also provide a characteristic expression profile for monitoring the DUOC-01 manufacturing process and potentially for measuring product potency. However, as CB CD14+ monocytes also promote remyelination, albeit less extensively than DUOC-01, gene products that are expressed by both DUOC-01 and by CB CD14+ cells may also play a role in the mechanism of action and potency of the cell product.

To further elucidate these mechanisms, expression of proteins corresponding to several of the transcripts expressed strongly or uniquely by DUOC-01 was examined. This is important because it was found previously, and also report here for the chemokines and metalloproteases, that relative transcript abundance does correlate with protein abundance in all cases. Taken together, the previous work and this study show that DUOC-01 cells constitutively secrete the following proteins that can affect brain remyelination and repair in different clinical contexts: 10 lysosomal hydrolases, IL-6, IL-10, TGFB, PDGFA, IGF1, KITLG, MMP7, MMP9, MMP12, CCL2, CCL4, CCL8, CCL13, CCL15, CCL20, CCL22, CCL23, CXCL8 and CXCL12. In addition, DUOC-01 cells also express receptors that, through signaling pathways, can generate additional downstream effectors affecting brain repair. At the protein level, these include several membrane proteins widely expressed by macrophage and also TREM2, a receptor with particularly important functions in brain macrophage. Several potential networks for enhancing remyelination are implied by these transcript and protein expression data. Some of these pathways were identified previously as being important in microglia that mediate remyelination in the cuprizone model. Most of the gene products discussed have multiple biological effects. Depending on the nature of an injury and the timing of when the gene products are mobilized, some of these products could potentially contribute to harmful inflammatory or pathological effects as well as reparative outcomes. DUOC-01 treatment enhanced remyelination in the cuprizone model, showed no toxicity in the preclinical safety studies required to obtain clearance to initiate clinical trial NCT02254863 and, caused no apparent adverse reactions in that ongoing trial. On the basis of this finding that DUOC-01 overexpressed transcripts for several matrix modifying proteases and secreted large amounts of MMP12 protein, it was suggested that DUOC-01 could regulate remyelination and repair by modifying the extracellular matrix. Activities of the brain matrix and matrix proteases in control of development and repair have been reviewed. FIG. 12 shows that transcripts for matrix modifying proteases MMP7, MMP12, CPE, MMP9, CPSK, CTSL, CTSB and NLN are abundant in DUOC-01. MMP7, MMP12 and CPE are not expressed by CD14+ monocytes; conversely CD14+ monocytes, but not DUOC-01, express MMP25, demonstrating specific regulation of this family of molecules. MMP2, MMP7, MMP9 and MMP12 proteins are released into the medium in substantial amounts during manufacture of DUOC-01. Significantly, DUOC-01, but not CD14+ monocytes, also over-expresses transcripts encoding A2M and TIMP3, the primary proteins that regulate the activity of matrix proteases. Furthermore, transcripts for matrix proteins constitute another of gene products that are expressed by DUOC-01. COL6A1 and COL6A2 transcripts are more than 1000-fold more abundant in DUOC-01 than in CD14+ monocytes, and SPP1, FN1 and SPARC are also highly over-expressed. Again, regulation of these matrix proteins appears to be specifically coordinated: CD14+ monocytes do not express the COL6A1, COL6A2, A2M or TIMP3 but do express THBS1 transcripts; DUOC-01 expresses the two collagens and protease inhibitors but not THBS1. Metalloproteases, including MMP9 and MMP12 that are secreted in abundance by DUOC-01, can affect synaptic plasticity and neural sprouting through effects on the matrix. Thus, these results suggest that several pathways coordinately regulated in DUOC-01 during manufacturing can degrade the extracellular matrix in a damaged brain and rebuild it by secreting new proteins. Consistent with this idea, transcripts for HS3STI1, a gene that encodes an enzyme that modifies heparin glycosaminoglycans; genes for signaling adherence receptors genes, VACAM1, ITGA6, ITGB8, NRP1, NRP2 and NEDD9 that regulate cell migration and interactions with the extracellular matrix; and complement component C1Qc that participates in synaptic remodeling are abundant in DUOC-01.

Another new result reported here is that DUOC-01 also secretes many CC- and CXC-type chemokines. Transcripts for chemokines CCL22 and CCL13 were uniquely expressed by DUOC-01; CCL2 and CCL4 transcripts were over-expressed relative to CD14+ monocytes; and CXCL8 transcripts were somewhat less strongly expressed in DUOC-01. CXCL12 was not differentially expressed. Immunoassay of culture supernatants showed that proteins corresponding to all these chemokines measured in the custom qPCR array as well as CCL8, CCL13, CCL15, CCL20 and CCL23 proteins accumulated at significant concentrations in culture supernatants during manufacturing. The relative amounts of each cytokine protein accumulating in the supernatants did not always reflect the relative abundance of transcripts. Collectively these secreted cytokines can potentially modulate the activity of many cell types bearing CCR1, CCR2, CCR3, CCR4, CCR5, CCR6, CXCR1, CXCR2, CXCR4 or CXCR7 receptors that are involved in brain inflammatory and repair reactions. Although expression of all these chemokines was up-regulated in DUOC-01, transcripts for chemokine receptors CCR2 and CX3CR1 could not be detected. In contrast, CD14+ monocytes expressed transcripts for these receptors. Both DUOC-01 and CD14+ monocytes express CXCR4. Some of the chemokines secreted by DUOC-01 enhance remyelination. Thus, CXCL12 (SPDF) protein controls migration of neural and oligodendrocyte progenitors bearing CXCR4 receptors to demyelinated areas and promotes myelination in animals and culture systems. It was previously shown that treatment with DUOC-01 drives proliferation and differentiation of oligodendrocyte progenitors and attributed this to secretion the activity of PDGFα, IGF1 and KITL proteins secreted by the cell product; CXCL12 secretion could augment this activity. Chemokines also attract microglia to lesions during remyelination processes. In one recent animal study, for example, astrocyte-generated IL-6 reduced local chemokine secretion, which, in turn, reduced microglial infiltration, debris removal through the microglial TREM2 receptor and finally remyelination. The chemokines involved included CCL4, major secretory product of DUOC-01. It was previously showed, and confirmed here, that IL-6 is a major, regulated secretory product of DUOC-01; this DUOC-01 could supplement astrocyte secretion of this cytokine. Finally, chemokine CXCL8 (IL8) is strongly angiogenic. VEGFA transcripts were also abundant in DUOC-01, suggesting that DUOC-01 can promote formation of new blood vessels following brain injury.

DUOC-01 could also modulate brain remyelination by TREM2-mediated phagocytic removal of dead cells and myelin debris from an injury site followed by signaling to glial cells. Evidence for the importance of the microglial activities including TREM2-mediated phagocytosis in brain repair in the cuprizone model and in human neurodegenerative conditions continues to accumulate. DUOC-01 cells are highly phagocytic in culture, and TREM1 is down-regulated and TREM2 upregulated during DUOC-01 manufacturing. Furthermore, DUOC-01 cells also express an array of transcripts related to lipid uptake, signaling and metabolism—activities associated with handling of myelin debris. These transcripts include lipid binding molecules APOE, APOC1 and COLEC112; lipid uptake receptors LRP5, LRP11 and LRP12; and lipid degrading LPL. Recent work has shown that APOE and other lipid particles are taken up by brain microglia through TREM2 and that other potentially neurotoxic molecules such as amyloid components can also be cleared by this mechanism. Additionally, with regard to modulation of myelination through lipid mediated reactions, transcripts encoding several enzymes responsible for synthesizing and converting prostaglandins from lipids (PTGDS, HPGDS, PTGES, PTGFRN, PTGR1 and PTGR2) are over-expressed in DUOC-01. Thus, DUOC-01 appears to be activated to handle lipids from degraded myelin in several ways that modulate the brain repair. The qPCR data imply other potential mechanisms for enhancing brain repair. Among these, the array data also suggest that DUOC-01 has characteristics of reparative, as opposed to pro-inflammatory, macrophage. DUOC-01 secretes substantial amounts of the immunosuppressive cytokine IL-10. The expression of NRP1, NRP2 transcripts by DUOC-01 is interesting in this regard as human monocytes that differentiate in environments leading to an M2 type polarization express these receptors. High expression of transcripts for three secreted proteins, CTH, GPX3 and, especially, SEPP1, that can enhance brain repair by regulating local redox potential was also detected.

From a manufacturing perspective, the changes in gene expression described here have allowed us to assign specific gene expression signatures that characterize CB CD14+ monocytes, the cells in CB that give rise to DUOC-01 during manufacturing, and the cells in the final cell product. Because the all the studied transcripts appear to be regulated coordinately during the first 2 weeks of the manufacturing process, because redundant probes for some repair pathways were included in the custom array and because accumulation of 16 secreted proteins could be detect in the supernatants of cultures by day 14, it was anticipate that smaller qPCR arrays along with assays of protein accumulation in the culture medium will become useful for in process testing and as potency assays to release product based on biological activity. Together these results suggest that it may be possible to shorten the manufacturing process provided functional characteristics are fully developed. However, as already noted, the relationship between the gene products measured for manufacturing purposes and the mechanism of action of DUOC-01 remains to be firmly established. Gene products that show large changes in expression during manufacturing were selected for analysis. Genes that show less extreme or even no changes in transcriptional regulation could contribute to the activity of DUOC-01 in enhancing remyelination. Finally, these results further support the hypothesis that cells in DUOC-01 arise from CD14+ monocytes present in cultured CB MNC. Thus, CB MNC and CB CD14+ monocytes respond to the procedures used to manufacture DUOC-01 regulate transcript abundance in similar ways and accumulate similar amounts of proteins in the culture medium with very similar time courses. Whole transcriptome analysis by microarrays confirmed that these cell products were highly similar. Manufacturing cell products from CB MNC, the method used to make the DUOC-01 cell product that is currently in the clinic, or from CB CD14+ monocytes, results cell products that are highly similar with regard to all the analytical metrics used.

In one respect, the high degree of similarity between cell products derived from MNCs and from purified CD14+ monocytes is surprising. The DUOC-01 population that emerges from cultured CB MNCs, unlike the cell product that arises from purified CD14+ monocytes, is exposed to many CB erythrocytes and dead and dying leucocytes. High concentrations of mature, non-nucleated erythrocytes in culture adversely influences the yield of DUOC-01 from manufacturing. During manufacturing, many dead CB cells are phagocytosed by the adherent cells that become part of the cell product. Human macrophage can be activated by different agents into many different activation states in which the cells express very different gene products. Phagocytosis of dead cells induces a unique macrophage activation state. Erythrocyte-derived heme can alter macrophage activation. Nevertheless, DUOC-01 derived from MNC and cell products derived from CD14+ monocytes are similar. CB monocytes and macrophage have been reported by many, but not all, workers to differ from adult peripheral blood monocytes with regard to their ability to be activated by toll-like receptor agonists and other agents. Fetal and adult monocytes also differ in response to several inflammatory stimuli. Of particular interest, human CB-derived macrophage does not become apoptotic after phagocytosis of bacteria or apoptotic leucocytes as readily as macrophage-derived from adult monocytes, and phagocytosis by the CB and adult monocyte derived macrophage results in elaboration of different cytokines. CB monocytes also differ from adult monocytes in their ability to phagocytose proteins that may be related to Alzheimer disease onset. These unique attributes of CB CD14+ monocytes are likely to determine the biological activities of the cell products derived from them.

The biological activities of DUOC-01 arise from rapid changes in expression of multiple genes in many pathways that promote remyelination. Many of these pathways are similar to pathways activated in microglia that regulate brain repair mechanisms. Expression of these activities is a response of CB CD14+ monocytes to the other components of CB MNCs, the culture conditions and the processes used to manufacture the cell product.

### Example 11: Hydrocortisone with DUOC-01 Doesn't Change its Viability, Phenotype or Potential Efficacy

As to the rationale for addition of hydrocortisone (HC) to DUOC-01 cell product, DUOC-01 is being used as an adjuvant treatment in an on-going clinical trial intended to protect CNS in patients undergoing hematopoietic stem cell transplant (HSCT) using cord blood units as a treatment for genetically determined CNS diseases. Some of the patients experienced self-limited fever and hypotension 2 to 6 hours after intrathecal injection of DUOC-01 manufactured from a second cord blood unit. This led to a decision of adding HC to the DUOC-01 formulation for the intrathecal injection. Although, HC was effective at managing the patients' acute inflammatory response, there was concern that HC could also reduce the efficacy of DUOC-01 cells. Therefore, it was critical to assess if HC could negatively influence the efficacy of DUOC-01 cells.

It was first tested if DUOC-01 cells incubated with HC showed any change in viability or phenotype. To do so, DUOC-01 cells were cultured via standard procedures from frozen cord blood units, then harvested on Day 21 and re-suspended in Ringer's solution. After initial count and viability analysis, the cell suspension was split into three conditions: (1) Ringer's solution, (2) 3 mg/ml HC, and (3) 25 ng/ml dexamethasone (Dex).

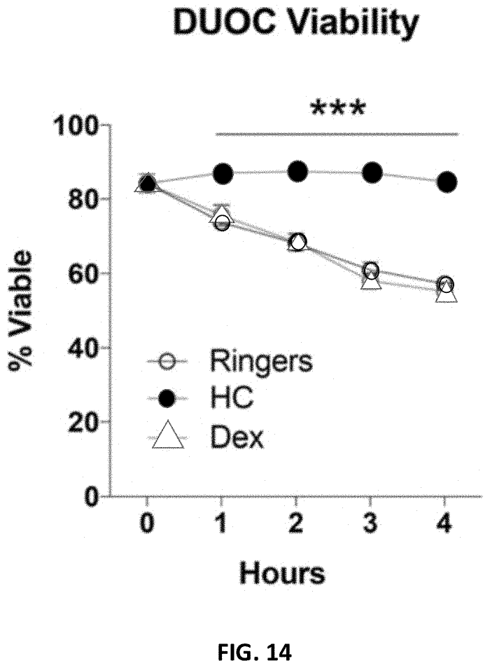

Based on previous stability studies, the cells under the aforementioned three conditions were incubated for 4 hours at room temperature and then characterized every hour for 4 hours. Viability was measured by AOPI using the Nexcelom Cellometer Auto 2000. The results show that the viability of DUOC-01 cells over time was consistently greater with cells incubated with HC compared to DUOC-01 cells incubated with dexamethasone or Ringer's solution (FIG. 14). That is, HC did not decrease viability of DUOC-01 cells in vitro; in fact, HC improved the viability DUOC-01 cells over time.

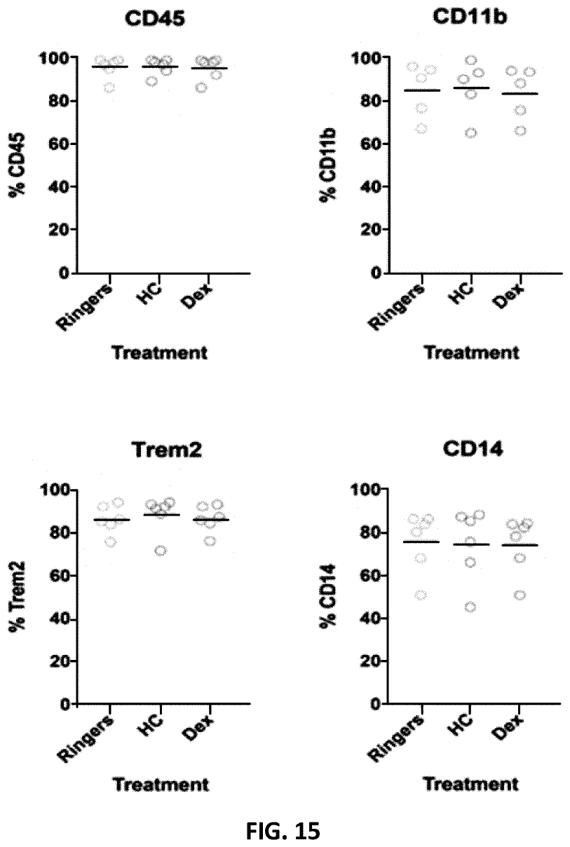

The results also show that HC did not change the expression level of common DUOC-01 cell surface markers (i.e., CD45, CD14, TREM2, and CD11b), which are used for standard characterization of DUOC-01 cells (see, FIG. 15). As illustrated therein, the cells under all three conditions mentioned above did not change the basic phenotype throughout the time course.

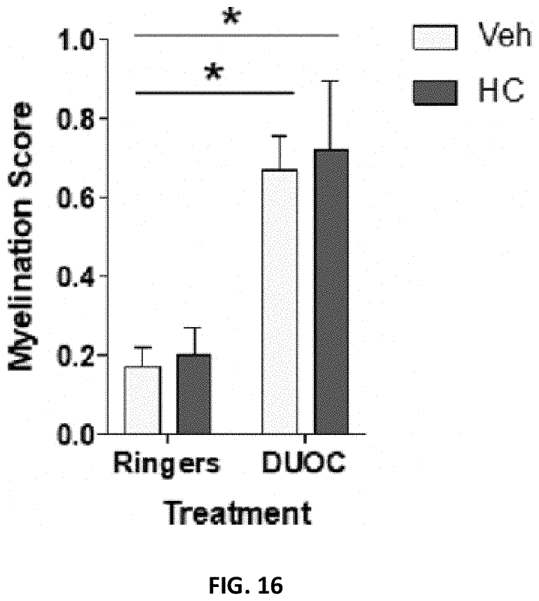

Next, it was tested if HC would impact the ability of DUOC-01 cells to promote remyelination in the standard murine cuprizone model of demyelination. To do so, myelination scores based on LFB-PAS staining of murine cuprizone model one week after treatment with Ringer's, Ringer's+HC, DUOC-01 cells, or DUOC-01 cells exposed to HC (DUOC-HC) (1×10⁵ cells injected) were presented on a bar graph (see, FIG. 16). Two independent, blinded readers scored coded LFB-PAS stained sections at the mid-line corpus callosum area, between zero and 3.

As already shown in the above examples, DUOC-01 accelerated remyelination, decreased gliosis, and reduced cellular infiltration in the brain of immune-incompetent mice exposed to the demyelinating agent, cuprizone. The remyelination effect of DUOC-01 was confirmed in this study in that the mice showed increased myelination in the corpus callosum area one week after DUOC-01 treatment when compared to Ringer's vehicle injected controls. The results further show that HC did not inhibit DUOC-01 cells from promoting remyelination. In fact, DUOC-01 cells exposed to HC promoted remyelination to the same extent as standard DUOC-01 cells (see, FIG. 16). That is, the data demonstrate that HC did not impact the function of DUOC-01 cells.

### Example 12: Hydrocortisone-Treated DUOC-01 (DUOC-HC) Ameliorates Experimental Autoimmune Encephalomyelitis (EAE)

To further elucidate the mechanism of action and investigate whether hydrocortisone-treated DUOC-01 (DUOC-HC) would be effective in other experimental models of demyelination, DUOC-01 was tested in experimental autoimmune encephalomyelitis (EAE), an animal model used to study multiple sclerosis (MS). In the event of an inflammatory response, it is possible that patients would receive a hydrocortisone (HC) injection at the time of infusing DUOC-01 cells. To mimic the practice in the clinic, DUOC-01 cells were first exposed to HC before they were administered to the EAE mice in this study.

EAE was induced in 8-week-old male C57BL/6 mice by injecting (subcutaneous) an emulsion of myelin oligodendrocyte glycoprotein peptide (MOG35-55) and complete Freund's adjuvant into the flanks. Each flank received injections of 100 μg of MOG35-55 for a total of 200 μg per mouse. 200 ng of pertussis toxin (intraperitoneal) were then injected to the mice at 2 and 24 hours later.

Using standard EAE clinical scoring system (see, Table 3), mice were monitored daily for paralysis. At the very early onset of the disease, i.e., with a clinical score 0.5-1.5, mice were allocated evenly into four groups to receive a single intra-cisterna magna injection into the cerebrospinal fluid. The four Groups were (1) Ringer's, (2) Ringer's+3 mg/mL HC, (3) 3×10⁵ DUOC-01 in Ringer's, and (4) 3×10⁵ DUOC-01 in Ringer's+3 mg/mL HC (DUOC-HC). For Group #4, DUOC-01 cells were incubated in Ringer's lactate solution with 3 mg/mL HC at a dilution of 1×10⁶ cells/ml for 2 hours at room temperature prior to injection. The DUOC-01 cells were administered at a dose of 3×10⁵ cells/mouse. The identity of the animals was blinded beyond this point and body weights and clinical scores were recorded daily for the remainder of the experiment.

TABLE 3
EAE Scoring Scale

| Score | Disease Symptoms |
|---|---|
| 0.0 | Healthy |
| 0.5 | Partial limp tail |
| 1.0 | Complete limp tail |
| 1.5 | Partial hind limb weakness |
| 2.0 | Complete hind limb weakness |
| 2.5 | Partial hind limb paralysis and one paralyzed leg |
| 3.0 | Complete hind limb paralysis and two paralyzed legs |
| 4.0 | Hind and fore limb paralysis |
| 5.0 | Mortality |

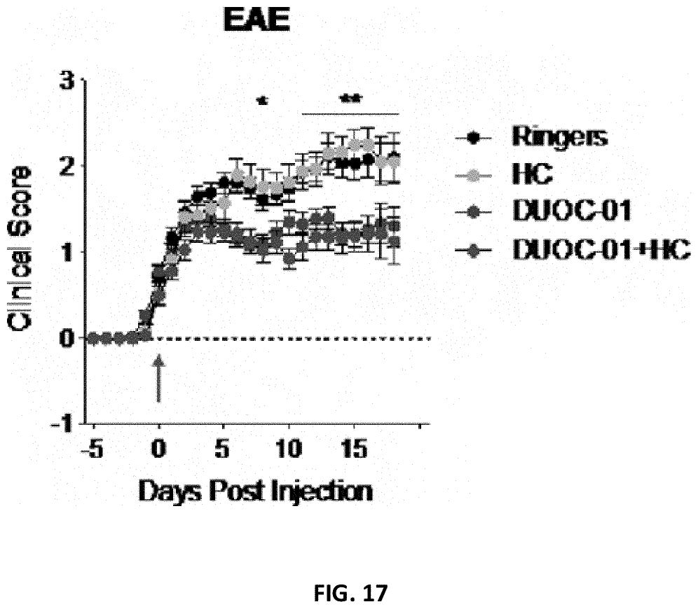

Compared with mice injected with Ringer's or HC+ Ringer's, mice injected with DUOC-01 (Group #3), or DUOC-01 incubated in HC (DUOC-HC) for 2 hours (Group #4), had decreased severity of the disease, as indicated by lower clinical scores based on severity of ascending paralysis (FIG. 17). The data suggest that the DUOC-01 cell therapy product could be beneficial in treating MS and other diverse neurological conditions with demyelination.

In conclusion, the addition of hydrocortisone (HC) did not change the DUOC-01 potency or stability in preclinical animal models as shown above and herein. On-going clinical trials also showed that patients treated with DUOC-01 formulated in HC (i.e., DUOC-HC) tolerated the DUOC-01 dosing without complications.

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

We claim:

1. A method of treating a demyelinating condition in a subject in need thereof, the method comprising:
administering to the subject a therapeutically effective amount of a DUOC-HC composition comprising a DUOC-01 cell product formulated in hydrocortisone (HC),
wherein the DUOC-01 cell product comprises cells derived from cord blood mononuclear cells, wherein such cells express one or more of CD45, CD11b, CD14, CD16, CD206, CD163, Iba1, HLA-DR, TREM 2, and iNOS macrophage or microglia markers; and wherein such cells secrete IL-6 and IL-10.

2. The method of claim 1, wherein the DUOC-01 cells are incubated in Ringer's lactate solution with hydrocortisone thereby obtaining the DUOC-HC composition.

3. The method of claim 1, wherein the demyelinating condition is multiple sclerosis, leukodystrophy, spinal cord injury, peripheral nerve damage, Parkinson's disease, amyotrophic lateral sclerosis (ALS), or Alzheimer's disease.

4. The method of claim 1, wherein the DUOC-01 cell product excludes cells expressing CD3.

5. The method of claim 1, wherein the DUOC-HC composition is administered via local tissue injection or intrathecally.

6. The method of claim 1, wherein the DUOC-HC composition is administered in a single dose or in multiple doses.

7. The method of claim 1, wherein the amount of the DUOC-HC composition administered is sufficient to provide about 1×10⁵ to about 1×10⁸ DUOC-01 cells.

8. The method of claim 1, further comprising:
exposing the cord blood mononuclear cells in a first culture medium to one or more factors selected from: platelet-derived growth factor (PDGF), neurotrophin-3 (NT-3), vascular endothelial growth factor (VEGF), and triiodothyronine (T₃); and at least one of serum or plasma for a period of time sufficient to obtain a DUOC-01 cell product;
isolating the DUOC-01 cell product; and
dissolving the DUOC-01 cell product in a pharmaceutically acceptable carrier to obtain the DUOC-HC composition.

9. The method of claim 8, wherein the exposing is to PDGF, NT-3, VEGF, T₃, and serum.

10. The method of claim 8, wherein the PDGF is present in a concentration of about 1 to about 10 ng/mL; the NT-3 is present in a concentration of about 0.1 to about 5 ng/mL; the VEGF is present in a concentration of about 1 to about 50 ng/mL; and the T₃ is present in a concentration of about 10 to about 100 ng/mL.

11. The method of claim 9, further comprising providing an additional amount of PDGF, NT-3, VEGF, T₃, and serum after 7 days and after 17 days.

12. The method of claim 9, further comprising providing an additional amount of PDGF, NT-3, and VEGF after 14 days.

13. The method of claim 8, wherein the period of time sufficient to obtain the DUOC-01 cell product is 21 days.

14. The method of claim 8, wherein the pharmaceutically acceptable carrier is Ringer's lactate solution with hydrocortisone.

15. A kit comprising:
a DUOC-HC composition comprising a DUOC-01 cell product formulated in hydrocortisone (HC), wherein DUOC-01 cell product comprises cells derived from cord blood mononuclear cells, wherein such cells express one or more of CD45, CD11b, CD14, CD16, CD206, CD163, Iba1, HLA-DR, TREM 2, and iNOS macrophage or microglia markers; and wherein such cells secrete IL-6 and IL-10; and
a label or instructions for administration of the DUOC-HC composition to treat a demyelinating condition.

16. The kit of claim 15, wherein the cells overexpress one or more of PDGFA, KITLG/SCF, IGF1, TREM2, MMP9, and MMP12 transcripts.

17. The kit of claim 15, wherein the amount of the DUOC-HC composition is sufficient to provide about 1×10⁵ to about 1×10⁸ DUOC-01 cells.

18. The kit of claim 15, wherein the DUOC-01 cell product is obtained by:
exposing the cord blood mononuclear cells in a first culture medium to one or more factors selected from: platelet-derived growth factor (PDGF), neurotrophin-3 (NT-3), vascular endothelial growth factor (VEGF), and triiodothyronine (T₃); and at least one of serum or plasma for a period of time sufficient to obtain a DUOC-01 cell product;
isolating the DUOC-01 cell product, and
dissolving the DUOC-01 cell product in a pharmaceutically acceptable carrier to obtain the DUOC-HC composition.

19. The kit of claim 18, wherein the pharmaceutically acceptable carrier is Ringer's lactate solution with hydrocortisone.

20. The kit of claim 15, wherein the demyelinating condition is multiple sclerosis, leukodystrophy, peripheral nerve disease, spinal cord injury, Parkinson's disease, amyotrophic lateral sclerosis (ALS), or Alzheimer's disease.
